(19) United States
(12) Patent Application Publication
(10) Pub. No.: US 2014/0256592 A1
(43) Pub. Date: Sep. 11, 2014

(54) DETERMINING RESPONSIVENESS OF AUTOIMMUNE PATIENTS TO DMARD TREATMENT

(71) Applicant: SEQUENTA, INC., South San FRancisco, CA (US)

(72) Inventor: Malek Faham, South San Franicisco, CA (US)

(21) Appl. No.: 14/350,516

(22) PCT Filed: Oct. 5, 2012
(86) PCT No.: PCT/US12/58989

§ 371 Date: Apr. 8, 2014

Related U.S. Application Data
(60) Provisional application No. 61/545,850, filed on Oct. 11, 2011.

(51) Int. Cl. C12Q 1/68

(52) U.S. Cl. CPC C12Q 1/6874; C12Q 1/686
USPC 506/9; 435/6.12

(57) ABSTRACT

The invention is directed to a method of screening patients suffering from an autoimmune disease for responsiveness to treatment with a disease modifying anti-rheumatic drug, or DMARD. In some embodiments the method of the invention comprises the steps of (a) measuring an IgH clonotype profile from B-cells in a sample of tissue affected by the autoimmune disease, the IgH clonotype profile including IgH clonotypes, IgG clonotypes, and IgD clonotypes; and (b) classifying a patient as being more likely to respond to DMARD treatment, whenever the patient has, with respect to reference levels characteristic of normal tissue, elevated IgH concentration, elevated IgG fraction, and reduced IgD fraction.

### CROSS-REFERENCE

This application claims the benefit of U.S. Provisional Patent Application No. 61/545,850, filed Oct. 11, 2011, which is herein incorporated by reference in its entirety.

### BACKGROUND OF THE INVENTION

Disease modifying anti-rheumatic drugs (or “DMARDs”) are given to autoimmune patients for a wide variety of autoimmune diseases in addition to rheumatoid diseases. The term DMARD originally meant a drug that affects biological measures such as erythrocyte sedimentation rate (ESR) and hemoglobin and autoantibody levels, or the like, but its current usage has come to mean a drug that reduces the rate of damage to bone and cartilage in an autoimmune disorder. DMARDs have been found both to produce durable symptomatic remissions and to delay or halt progression of such damage. Moreover, when responsiveness declines to a particular DMARD, a more potent DMARD may be substituted to restore the beneficial effects. DMARDs are used to treat rheumatoid arthritis, Crohn's disease, systemic lupus erythematosis, immune thrombocytopenic purpura, myasthenia gravis, and other autoimmune conditions. Thus, it would be valuable to have an early measure of patient responsiveness to treatments with DMARDs to ensure selection of the best drug for a patient's condition.

Profiles of nucleic acids encoding immune molecules, such as T cell or B cell receptors, or their components, contain a wealth of information on the state of health or disease of an organism, so that the use of such profiles as diagnostic or prognostic indicators has been proposed for a wide variety of conditions, including autoimmune conditions e.g. Faham and Willis, U.S. patent publication 2010/0151471 and 2011/0207134; Freeman et al, Genome Research, 19: 1817-1824 (2009); Boyd et al, Sci. Transl. Med., 1(12): 12ra23 (2009); He et al, Oncotarget (Mar. 8, 2011). Such sequence-based profiles are capable of much greater sensitivity than approaches based on size distributions of amplified CDR-encoding regions, sequence sampling by microarrays, hybridization kinetics curves from PCR amplicons, or other approaches, e.g. Morley et al, U.S. Pat. No. 5,418,134; van Dongen et al, Leukemia, 17: 2257-2317 (2003); Ogle et al, Nucleic Acids Research, 31: e139 (2003); Wang et al, BMC Genomics, 8: 329 (2007); Baum et al, Nature Methods, 3(11): 895-901 (2006).

In view of the importance of immune changes to a wide variety of treatments, including DMARD treatments for autoimmune diseases, it would be highly desirable if measures were available based on sequence profiles that could readily be correlated to states of health or disease and/or likelihood of treatment success.

### SUMMARY OF THE INVENTION

The present invention is drawn to methods for producing sequence-based profiles of complex nucleic acid populations. The invention is exemplified in a number of implementations and applications, some of which are summarized below and throughout the specification.

In one aspect, the invention is directed to a method of screening patients suffering from an autoimmune disease for responsiveness to treatment with a disease modifying anti-rheumatic drug (DMARD) comprising the steps of: (a) determining an IgH clonotype profile from B-cells in a sample of tissue affected by the autoimmune disease, the IgH clonotype profile including IgH clonotypes, IgG clonotypes, and IgD clonotypes; and (b) classifying a patient as being more likely to respond to DMARD treatment, whenever the patient has, with respect to reference levels characteristic of normal tissue, elevated IgH concentration, elevated IgG fraction, and reduced IgD fraction.

In another aspect, the invention includes a method of determining responsiveness of a patient having psoriatic arthritis to treatment with a disease modifying anti-rheumatic drug (DMARD) comprising the steps of: (a) determining an IgH clonotype profile from B-cells in a sample of synovial fluid, the IgH clonotype profile including IgH clonotypes, IgG clonotypes, and IgD clonotypes; and (b) classifying a patient as being more likely to respond to DMARD treatment, whenever the patient has, with respect to reference levels characteristic of normal tissue, elevated IgH concentration, elevated IgG fraction, and reduced IgD fraction. In some embodiments of the foregoing aspect, an IgH clonotype profile indicates at least three of the following conditions hold for said responsive patient: with respect to reference levels characteristic of peripheral blood mononuclear cells of said responsive patient, (a) elevated IgH concentration, (b) elevated IgG fraction, (c) reduced IgD fraction, (d) reduced IgD diversity, (e) reduced IgM diversity, and (f) elevated IgM somatic mutation rate.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the invention.

### BRIEF DESCRIPTION OF THE DRAWINGS

The novel features of the invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIG. 1A illustrates a PCR scheme for generating three sequencing templates from an IgH chain in a single reaction. FIGS. 1B-1C illustrates a PCR scheme for generating three sequencing templates from an IgH chain in three separate reactions after which the resulting amplicons are combined for a secondary PCR to add P5 and P7 primer binding sites. FIG. 1D illustrates the locations of sequence reads generated for an IgH chain. FIG. 1E illustrates the use of the codon structure of V and J regions to improve base calls in the NDN region.

FIGS. 2A-2C show a two-staged PCR scheme for amplifying TCRβ genes.

FIG. 2D illustrates details of determining a nucleotide sequence of the PCR product of FIG. 2C. FIG. 2E illustrates details of another embodiment of determining a nucleotide sequence of the PCR product of FIG. 2C.

FIG. 3 shows relative per base error rates for different segments (J, 300; V2, 302; V1, 304) of the sequence reads used to construct clonotypes.

FIG. 4 shows the total mapped read counts for different experiments.

FIG. 5 shows total cloned read counts as a function of cloning frequency for PBMC samples (light colored boxes, 500) and SF samples (dark colored boxes, 502).

FIG. 6 shows fraction of reads in frame for different experiments.

FIGS. 7A-7B show TCRβ repertoire replicability in repeat measurements for synovial fluid samples (7A) and PBMC samples (7B).

FIGS. 8A-8C show TCRβ repertoire comparisons in the same patient: 8A: synovial fluid samples taken about one month apart, 8B: PBMC samples taken about one month apart, and 8C: PBMC (x-axis) and synovial fluid (y-axis) taken at the same time.

FIGS. 9A-9B show the distributions of clonotype frequencies within a PBMC (9A) clonotype profile and an SF (9B) clonotype profile.

FIG. 10 shows total IgH mapped reads versus experiment for PBMCs (light boxes) and SF (dark boxes).

FIG. 11 shows total read counts versus cloning frequency for PBMCs (light boxes, 1100) and SF (dark boxes, 1102).

FIG. 12 shows the fraction of IgH sequences that are in-frame for PBMCs (light boxes, 1200) and SF (dark boxes, 1202).

FIGS. 13A-13B show IgH repertoire replicability in repeat measurements for synovial fluid samples (13A) and PBMC samples (13B).

FIGS. 14A-14C show IgH repertoire comparisons in the same patient: 14A: synovial fluid samples from patient 001 taken about two months apart, 14B: SF samples from patient 002 taken about two months apart, and 14C: SF samples from patient 003 taken about two months apart.

FIGS. 15A-15B show repertoire parameters distinguishing PBMC clonotypes profiles from SF clonotype profiles. FIG. 15A shows IgG fraction versus IgD fraction. FIG. 15B shows diversity within IgM and IgD classes. Light colored boxes (1504 and 1506) are PBMC data points and dark colored boxes (1508 and 1510) are SF data points.

### DETAILED DESCRIPTION OF THE INVENTION

The practice of the present invention may employ, unless otherwise indicated, conventional techniques and descriptions of molecular biology (including recombinant techniques), bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; and Molecular Cloning: A Laboratory Manual (all from Cold Spring Harbor Laboratory Press); Gusfield, Algorithms on Strings, Trees, and Sequences (Cambridge University Press, 1997); and the like.

The invention is directed to method for determining the responsiveness of an autoimmune patient to treatment by a DMARD. That is, the invention is directed to methods for screening for autoimmune patients who may be responsive to treatment by a DMARD. In accordance with the invention, a patient's responsiveness is determined from information derived from one or more clonotype profiles of T cells or B cells in a disease-affected tissue. DMARDs include, but are not limited to, tumor necrosis factor (TNF) inhibitors, purine synthesis inhibitors, calcineurin inhibitors, arachidonate 5-lipoxygenase (5-LO) inhibitors, and the like. More specifically, DMARDs include, but are not limited to, the following drugs: adalimumab, azathioprine, chloroquine, hydroxychloroquine, ciclosporin, D-penicillamine, etanercept, golimumab, gold salts, infliximab, leflunomide, methotrexate, minocycline, rituximab, sulfasalazine, and the like. Clonotype profiles are measured as taught in Faham and Willis, U.S. patent publication US2011/0207134, which is incorporated herein by reference in its entirety. Briefly, in one aspect, a sequence-based clonotype profile of an individual is obtained using the following steps: (a) obtaining a nucleic acid sample from T-cells and/or B-cells of the individual; (b) spatially isolating individual molecules derived from such nucleic acid sample, the individual molecules comprising nested sets of templates each generated from a nucleic acid in the sample and each containing a somatically rearranged region or a portion thereof, each nested set being capable of producing a plurality of sequence reads each extending in the same direction and each starting from a different position on the nucleic acid from which the nested set was generated; (c) sequencing said spatially isolated individual molecules; and (d) determining abundances of different sequences of the nucleic acid molecules from the nucleic acid sample to generate the clonotype profile. In one embodiment, the step of sequencing includes producing a plurality of sequence reads for each of the nested sets. In another embodiment, each of the somatically rearranged regions comprise a V region and a J region, and each of the plurality of sequence reads starts from a different position in the V region and extends in the direction of its associated J region. In another embodiment, the step of sequencing comprises bidirectionally sequencing each of the spatially isolated individual molecules to produce at least one forward sequence read and at least one reverse sequence read. Further to the latter embodiment, at least one of the forward sequence reads and at least one of the reverse sequence reads have an overlap region such that bases of such overlap region are determined by a reverse complementary relationship between such sequence reads. In still another embodiment, each of the somatically rearranged regions comprise a V region and a J region and the step of sequencing further includes determining a sequence of each of the individual nucleic acid molecules from one or more of its forward sequence reads and at least one reverse sequence read starting from a position in a J region and extending in the direction of its associated V region. In another embodiment, individual molecules comprise nucleic acids selected from the group consisting of complete IgH molecules, incomplete IgH molecules, complete IgK complete, IgK inactive molecules, TCRβ molecules, TCRγ molecules, complete TCRδ molecules, and incomplete TCRδ molecules. In another embodiment, the step of sequencing comprises generating the sequence reads having monotonically decreasing quality scores. Further to the latter embodiment, monotonically decreasing quality scores are such that the sequence reads have error rates no better than the following: 0.2 percent of sequence reads contain at least one error in base positions 1 to 50, 0.2 to 1.0 percent of sequence reads contain at least one error in positions 51-75, 0.5 to 1.5 percent of sequence reads contain at least one error in positions 76-100.

### Amplification of Nucleic Acid Populations

Amplicons of target populations of nucleic acids may be generated by a variety of amplification techniques. In one aspect of the invention, multiplex PCR is used to amplify members of a mixture of nucleic acids, particularly mixtures comprising recombined immune molecules such as T cell receptors, B cell receptors, or portions thereof. Guidance for carrying out multiplex PCRs of such immune molecules is found in the following references, which are incorporated by reference: Morley, U.S. Pat. No. 5,296,351; Gorski, U.S. Pat. No. 5,837,447; Dau, U.S. Pat. No. 6,087,096; Von Dongen et al, U.S. patent publication 2006/0234234; European patent publication EP 1544308B1; and the like.

After amplification of DNA from the genome (or amplification of nucleic acid in the form of cDNA by reverse transcribing RNA), the individual nucleic acid molecules can be isolated, optionally re-amplified, and then sequenced individually. Exemplary amplification protocols may be found in van Dongen et al, Leukemia, 17: 2257-2317 (2003) or van Dongen et al, U.S. patent publication 2006/0234234, which is incorporated by reference. Briefly, an exemplary protocol is as follows: Reaction buffer: ABI Buffer II or ABI Gold Buffer (Life Technologies, San Diego, Calif.); 50 μL final reaction volume; 100 ng sample DNA; 10 pmol of each primer (subject to adjustments to balance amplification as described below); dNTPs at 200 μM final concentration; MgCl₂ at 1.5 mM final concentration (subject to optimization depending on target sequences and polymerase); Taq polymerase (1-2 U/tube); cycling conditions: preactivation 7 min at 95° C.; annealing at 60° C.; cycling times: 30 s denaturation; 30 s annealing; 30 s extension. Polymerases that can be used for amplification in the methods of the invention are commercially available and include, for example, Taq polymerase, AccuPrime polymerase, or Pfu. The choice of polymerase to use can be based on whether fidelity or efficiency is preferred.

Real time PCR, picogreen staining, nanofluidic electrophoresis (e.g. LabChip) or UV absorption measurements can be used in an initial step to judge the functional amount of amplifiable material.

In one aspect, multiplex amplifications are carried out so that relative amounts of sequences in a starting population are substantially the same as those in the amplified population, or amplicon. That is, multiplex amplifications are carried out with minimal amplification bias among member sequences of a sample population. In one embodiment, such relative amounts are substantially the same if each relative amount in an amplicon is within five fold of its value in the starting sample. In another embodiment, such relative amounts are substantially the same if each relative amount in an amplicon is within two fold of its value in the starting sample. As discussed more fully below, amplification bias in PCR may be detected and corrected using conventional techniques so that a set of PCR primers may be selected for a predetermined repertoire that provide unbiased amplification of any sample.

In regard to many repertoires based on TCR or BCR sequences, a multiplex amplification optionally uses all the V segments. The reaction is optimized to attempt to get amplification that maintains the relative abundance of the sequences amplified by different V segment primers. Some of the primers are related, and hence many of the primers may “cross talk,” amplifying templates that are not perfectly matched with it. The conditions are optimized so that each template can be amplified in a similar fashion irrespective of which primer amplified it. In other words if there are two templates, then after 1,000 fold amplification both templates can be amplified approximately 1,000 fold, and it does not matter that for one of the templates half of the amplified products carried a different primer because of the cross talk. In subsequent analysis of the sequencing data the primer sequence is eliminated from the analysis, and hence it does not matter what primer is used in the amplification as long as the templates are amplified equally.

In one embodiment, amplification bias may be avoided by carrying out a two-stage amplification (as described in Faham and Willis, cited above) wherein a small number of amplification cycles are implemented in a first, or primary, stage using primers having tails non-complementary with the target sequences. The tails include primer binding sites that are added to the ends of the sequences of the primary amplicon so that such sites are used in a second stage amplification using only a single forward primer and a single reverse primer, thereby eliminating a primary cause of amplification bias. Preferably, the primary PCR will have a small enough number of cycles (e.g. 5-10) to minimize the differential amplification by the different primers. The secondary amplification is done with one pair of primers and hence the issue of differential amplification is minimal. One percent of the primary PCR is taken directly to the secondary PCR. Thirty-five cycles (equivalent to ˜28 cycles without the 100 fold dilution step) used between the two amplifications were sufficient to show a robust amplification irrespective of whether the breakdown of cycles were: one cycle primary and 34 secondary or 25 primary and 10 secondary. Even though ideally doing only 1 cycle in the primary PCR may decrease the amplification bias, there are other considerations. One aspect of this is representation. This plays a role when the starting input amount is not in excess to the number of reads ultimately obtained. For example, if 1,000,000 reads are obtained and starting with 1,000,000 input molecules then taking only representation from 100,000 molecules to the secondary amplification would degrade the precision of estimating the relative abundance of the different species in the original sample. The 100 fold dilution between the 2 steps means that the representation is reduced unless the primary PCR amplification generated significantly more than 100 molecules. This indicates that a minimum 8 cycles (256 fold), but more comfortably 10 cycle (˜1,000 fold), may be used. The alternative to that is to take more than 1% of the primary PCR into the secondary but because of the high concentration of primer used in the primary PCR, a big dilution factor can be used to ensure these primers do not interfere in the amplification and worsen the amplification bias between sequences. Another alternative is to add a purification or enzymatic step to eliminate the primers from the primary PCR to allow a smaller dilution of it. In this example, the primary PCR was 10 cycles and the second 25 cycles.

### Generating Sequence Reads for Clonotypes

Any high-throughput technique for sequencing nucleic acids can be used in the method of the invention. Preferably, such technique has a capability of generating in a cost-effective manner a volume of sequence data from which at least 1000 clonotypes can be determined, and preferably, from which at least 10,000 to 1,000,000 clonotypes can be determined. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches can thus be used to study the repertoire of T-cell receptor (TCR) and/or B-cell receptor (BCR). In one aspect of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm². In one embodiment, sequencing chemistries are employed having relatively high error rates. In such embodiments, the average quality scores produced by such chemistries are monotonically declining functions of sequence read lengths. In one embodiment, such decline corresponds to 0.5 percent of sequence reads have at least one error in positions 1-75; 1 percent of sequence reads have at least one error in positions 76-100; and 2 percent of sequence reads have at least one error in positions 101-125.

In one aspect, a sequence-based clonotype profile of an individual is obtained using the following steps: (a) obtaining a nucleic acid sample from T-cells and/or B-cells of the individual; (b) spatially isolating individual molecules derived from such nucleic acid sample, the individual molecules comprising at least one template generated from a nucleic acid in the sample, which template comprises a somatically rearranged region or a portion thereof, each individual molecule being capable of producing at least one sequence read; (c) sequencing said spatially isolated individual molecules; and (d) determining abundances of different sequences of the nucleic acid molecules from the nucleic acid sample to generate the clonotype profile. In one embodiment, each of the somatically rearranged regions comprise a V region and a J region. In another embodiment, the step of sequencing comprises bidirectionally sequencing each of the spatially isolated individual molecules to produce at least one forward sequence read and at least one reverse sequence read. Further to the latter embodiment, at least one of the forward sequence reads and at least one of the reverse sequence reads have an overlap region such that bases of such overlap region are determined by a reverse complementary relationship between such sequence reads. In still another embodiment, each of the somatically rearranged regions comprise a V region and a J region and the step of sequencing further includes determining a sequence of each of the individual nucleic acid molecules from one or more of its forward sequence reads and at least one reverse sequence read starting from a position in a J region and extending in the direction of its associated V region. In another embodiment, individual molecules comprise nucleic acids selected from the group consisting of complete IgH molecules, incomplete IgH molecules, complete IgK complete, IgK inactive molecules, TCRβ molecules, TCRγ molecules, complete TCRδ molecules, and incomplete TCRδ molecules. In another embodiment, the step of sequencing comprises generating the sequence reads having monotonically decreasing quality scores. Further to the latter embodiment, monotonically decreasing quality scores are such that the sequence reads have error rates no better than the following: 0.2 percent of sequence reads contain at least one error in base positions 1 to 50, 0.2 to 1.0 percent of sequence reads contain at least one error in positions 51-75, 0.5 to 1.5 percent of sequence reads contain at least one error in positions 76-100. In another embodiment, the above method comprises the following steps: (a) obtaining a nucleic acid sample from T-cells and/or B-cells of the individual; (b) spatially isolating individual molecules derived from such nucleic acid sample, the individual molecules comprising nested sets of templates each generated from a nucleic acid in the sample and each containing a somatically rearranged region or a portion thereof, each nested set being capable of producing a plurality of sequence reads each extending in the same direction and each starting from a different position on the nucleic acid from which the nested set was generated; (c) sequencing said spatially isolated individual molecules; and (d) determining abundances of different sequences of the nucleic acid molecules from the nucleic acid sample to generate the clonotype profile. In one embodiment, the step of sequencing includes producing a plurality of sequence reads for each of the nested sets. In another embodiment, each of the somatically rearranged regions comprise a V region and a J region, and each of the plurality of sequence reads starts from a different position in the V region and extends in the direction of its associated J region.

In one aspect, for each sample from an individual, the sequencing technique used in the methods of the invention generates sequences of least 1000 clonotypes per run; in another aspect, such technique generates sequences of at least 10,000 clonotypes per run; in another aspect, such technique generates sequences of at least 100,000 clonotypes per run; in another aspect, such technique generates sequences of at least 500,000 clonotypes per run; and in another aspect, such technique generates sequences of at least 1,000,000 clonotypes per run. In still another aspect, such technique generates sequences of between 100,000 to 1,000,000 clonotypes per run per individual sample.

The sequencing technique used in the methods of the provided invention can generate about 30 bp, about 40 bp, about 50 bp, about 60 bp, about 70 bp, about 80 bp, about 90 bp, about 100 bp, about 110, about 120 bp per read, about 150 bp, about 200 bp, about 250 bp, about 300 bp, about 350 bp, about 400 bp, about 450 bp, about 500 bp, about 550 bp, or about 600 bp per read.

### Clonotype Determination from Sequence Data

Constructing clonotypes from sequence read data depends in part on the sequencing method used to generate such data, as the different methods have different expected read lengths and data quality. In one approach, a Solexa sequencer is employed to generate sequence read data for analysis as described in Faham and Willis, cited above. In one embodiment, a sample is obtained that provides at least 0.5-1.0×10⁶ lymphocytes to produce at least 1 million template molecules, which after optional amplification may produce a corresponding one million or more clonal populations of template molecules (or clusters). For most high throughput sequencing approaches, including the Solexa approach, such over sampling at the cluster level is desirable so that each template sequence is determined with a large degree of redundancy to increase the accuracy of sequence determination. For Solexa-based implementations, preferably the sequence of each independent template is determined 10 times or more. For other sequencing approaches with different expected read lengths and data quality, different levels of redundancy may be used for comparable accuracy of sequence determination. Those of ordinary skill in the art recognize that the above parameters, e.g. sample size, redundancy, and the like, are design choices related to particular applications.

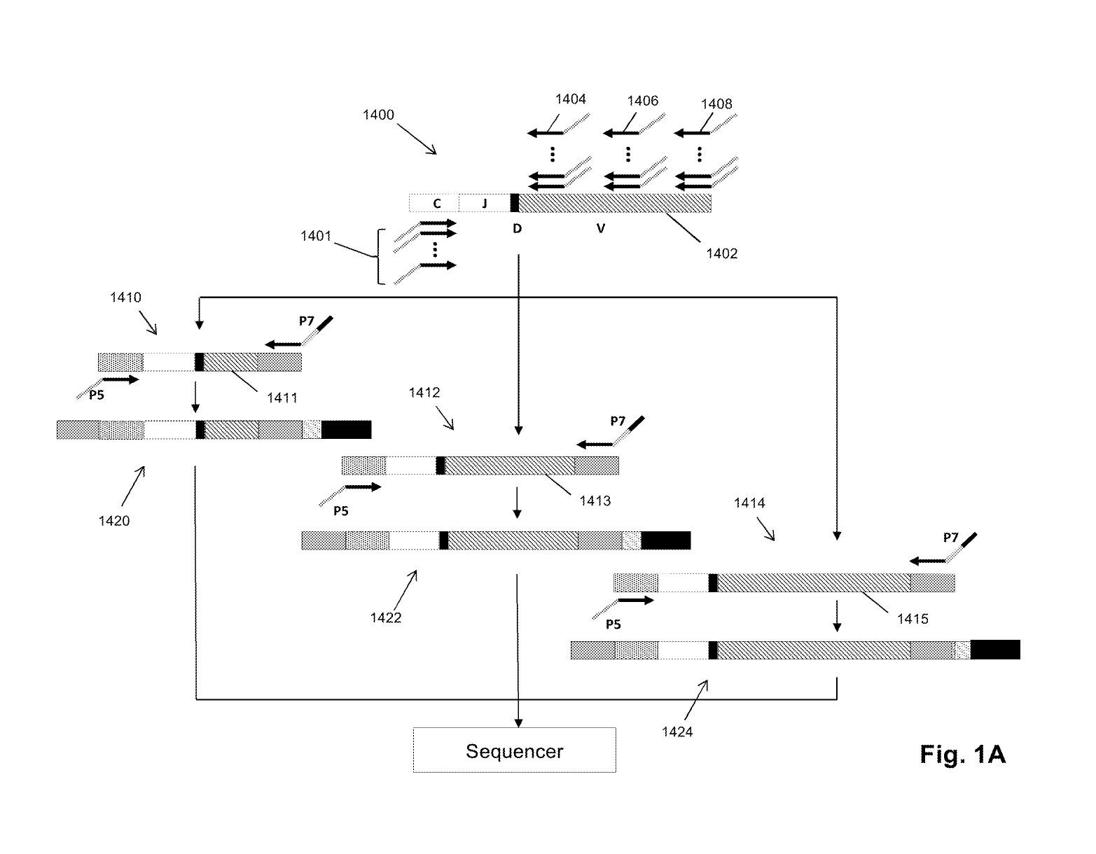

In one aspect of the invention, sequences of clonotypes (including but not limited to those derived from IgH, TCRα, TCRβ, TCRγ, TCRδ, and/or IgLκ (IgK)) may be determined by combining information from one or more sequence reads, for example, along the V(D)J regions of the selected chains. In another aspect, sequences of clonotypes are determined by combining information from a plurality of sequence reads. Such pluralities of sequence reads may include one or more sequence reads along a sense strand (i.e. “forward” sequence reads) and one or more sequence reads along its complementary strand (i.e. “reverse” sequence reads). When multiple sequence reads are generated along the same strand, separate templates are first generated by amplifying sample molecules with primers selected for the different positions of the sequence reads. This concept is illustrated in FIG. 1A where primers (1404, 1406 and 1408) are employed to generate amplicons (1410, 1412, and 1414, respectively) in a single reaction. Such amplifications may be carried out in the same reaction or in separate reactions. In one aspect, whenever PCR is employed, separate amplification reactions are used for generating the separate templates which, in turn, are combined and used to generate multiple sequence reads along the same strand. This latter approach is preferable for avoiding the need to balance primer concentrations (and/or other reaction parameters) to ensure equal amplification of the multiple templates (sometimes referred to herein as “balanced amplification” or “unbias amplification”). The generation of templates in separate reactions is illustrated in FIGS. 1B-1C. There a sample containing IgH (1400) is divided into three portions (1470, 1472, and 1474) which are added to separate PCRs using J region primers (1401) and V region primers (1404, 1406, and 1408, respectively) to produce amplicons (1420, 1422 and 1424, respectively). The latter amplicons are then combined (1478) in secondary PCR (1480) using P5 and P7 primers to prepare the templates (1482) for bridge PCR and sequencing on an Illumina GA sequencer, or like instrument.

Sequence reads of the invention may have a wide variety of lengths, depending in part on the sequencing technique being employed. For example, for some techniques, several trade-offs may arise in its implementation, for example, (i) the number and lengths of sequence reads per template and (ii) the cost and duration of a sequencing operation. In one embodiment, sequence reads are in the range of from 20 to 400 nucleotides; in another embodiment, sequence reads are in a range of from 30 to 200 nucleotides; in still another embodiment, sequence reads are in the range of from 30 to 120 nucleotides. In one embodiment, 1 to 4 sequence reads are generated for determining the sequence of each clonotype; in another embodiment, 2 to 4 sequence reads are generated for determining the sequence of each clonotype; and in another embodiment, 2 to 3 sequence reads are generated for determining the sequence of each clonotype. In the foregoing embodiments, the numbers given are exclusive of sequence reads used to identify samples from different individuals. The lengths of the various sequence reads used in the embodiments described below may also vary based on the information that is sought to be captured by the read; for example, the starting location and length of a sequence read may be designed to provide the length of an NDN region as well as its nucleotide sequence; thus, sequence reads spanning the entire NDN region are selected. In other aspects, one or more sequence reads that in combination (but not separately) encompass a D and/or NDN region are sufficient.

In another aspect of the invention, sequences of clonotypes are determined in part by aligning sequence reads to one or more V region reference sequences and one or more J region reference sequences, and in part by base determination without alignment to reference sequences, such as in the highly variable NDN region. A variety of alignment algorithms may be applied to the sequence reads and reference sequences. For example, guidance for selecting alignment methods is available in Batzoglou, Briefings in Bioinformatics, 6: 6-22 (2005), which is incorporated by reference. In one aspect, whenever V reads or C reads (as mentioned above) are aligned to V and J region reference sequences, a tree search algorithm is employed, e.g. as described generally in Gusfield (cited above) and Cormen et al, Introduction to Algorithms, Third Edition (The MIT Press, 2009).

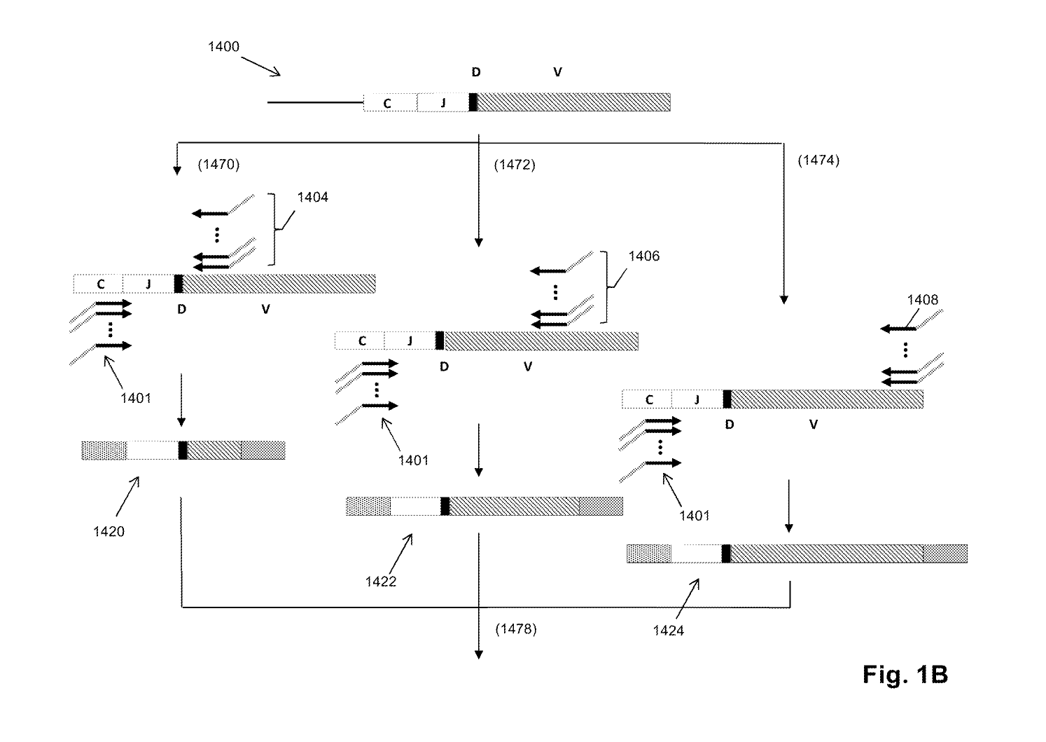

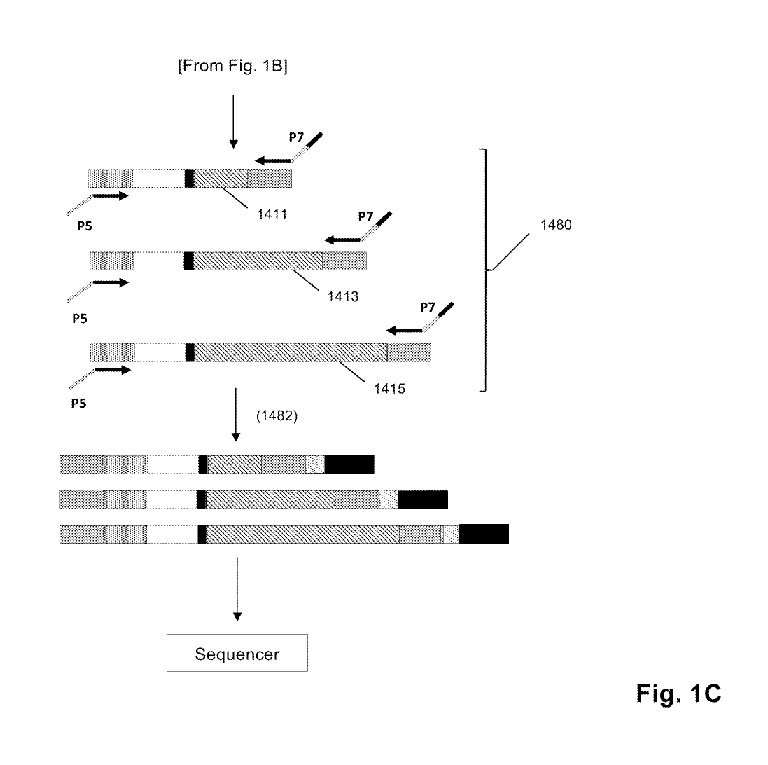

In another aspect, an end of at least one forward read and an end of at least one reverse read overlap in an overlap region (e.g. 2308 in FIG. 2D), so that the bases of the reads are in a reverse complementary relationship with one another. Thus, for example, if a forward read in the overlap region is “5′-acgttgc”, then a reverse read in a reverse complementary relationship is “5′-gcaacgt” within the same overlap region. In one aspect, bases within such an overlap region are determined, at least in part, from such a reverse complementary relationship. That is, a likelihood of a base call (or a related quality score) in a prospective overlap region is increased if it preserves, or is consistent with, a reverse complementary relationship between the two sequence reads. In one aspect, clonotypes of TCRβ and IgH chains (illustrated in FIG. 2D) are determined by at least one sequence read starting in its J region and extending in the direction of its associated V region (referred to herein as a “C read” (2304)) and at least one sequence read starting in its V region and extending in the direction of its associated J region (referred to herein as a “V read” (2306)). Overlap region (2308) may or may not encompass the NDN region (2315) as shown in FIG. 2D. Overlap region (2308) may be entirely in the J region, entirely in the NDN region, entirely in the V region, or it may encompass a J region-NDN region boundary or a V region-NDN region boundary, or both such boundaries (as illustrated in FIG. 2D). Typically, such sequence reads are generated by extending sequencing primers, e.g. (2302) and (2310) in FIG. 2D, with a polymerase in a sequencing-by-synthesis reaction, e.g. Metzger, Nature Reviews Genetics, 11: 31-46 (2010); Fuller et al, Nature Biotechnology, 27: 1013-1023 (2009). The binding sites for primers (2302) and (2310) are predetermined, so that they can provide a starting point or anchoring point for initial alignment and analysis of the sequence reads. In one embodiment, a C read is positioned so that it encompasses the D and/or NDN region of the TCRβ or IgH chain and includes a portion of the adjacent V region, e.g. as illustrated in FIGS. 2D and 2E. In one aspect, the overlap of the V read and the C read in the V region is used to align the reads with one another. In other embodiments, such alignment of sequence reads is not necessary, e.g. with TCRβ chains, so that a V read may only be long enough to identify the particular V region of a clonotype. This latter aspect is illustrated in FIG. 2E. Sequence read (2330) is used to identify a V region, with or without overlapping another sequence read, and another sequence read (2332) traverses the NDN region and is used to determine the sequence thereof. Portion (2334) of sequence read (2332) that extends into the V region is used to associate the sequence information of sequence read (2332) with that of sequence read (2330) to determine a clonotype. For some sequencing methods, such as base-by-base approaches like the Solexa sequencing method, sequencing run time and reagent costs are reduced by minimizing the number of sequencing cycles in an analysis. Optionally, as illustrated in FIG. 2D, amplicon (2300) is produced with sample tag (2312) to distinguish between clonotypes originating from different biological samples, e.g. different patients. Sample tag (2312) may be identified by annealing a primer to primer binding region (2316) and extending it (2314) to produce a sequence read across tag (2312), from which sample tag (2312) is decoded.

The IgH chain is more challenging to analyze than TCRβ chain because of at least two factors: i) the presence of somatic mutations makes the mapping or alignment more difficult, and ii) the NDN region is larger so that it is often not possible to map a portion of the V segment to the C read. In one aspect of the invention, this problem is overcome by using a plurality of primer sets for generating V reads, which are located at different locations along the V region, preferably so that the primer binding sites are nonoverlapping and spaced apart, and with at least one primer binding site adjacent to the NDN region, e.g. in one embodiment from 5 to 50 bases from the V-NDN junction, or in another embodiment from 10 to 50 bases from the V-NDN junction. The redundancy of a plurality of primer sets minimizes the risk of failing to detect a clonotype due to a failure of one or two primers having binding sites affected by somatic mutations. In addition, the presence of at least one primer binding site adjacent to the NDN region makes it more likely that a V read will overlap with the C read and hence effectively extend the length of the C read. This allows for the generation of a continuous sequence that spans all sizes of NDN regions and that can also map substantially the entire V and J regions on both sides of the NDN region. Embodiments for carrying out such a scheme are illustrated in FIGS. 1A and 1D. In FIG. 1A, a sample comprising IgH chains (1400) are sequenced by generating a plurality amplicons for each chain by amplifying the chains with a single set of J region primers (1401) and a plurality (three shown) of sets of V region (1402) primers (1404, 1406, 1408) to produce a plurality of nested amplicons (e.g., 1410, 1412, 1414) all comprising the same NDN region and having different lengths encompassing successively larger portions (1411, 1413, 1415) of V region (1402). Members of a nested set may be grouped together after sequencing by noting the identify (or substantial identity) of their respective NDN, J and/or C regions, thereby allowing reconstruction of a longer V(D)J segment than would be the case otherwise for a sequencing platform with limited read length and/or sequence quality. In one embodiment, the plurality of primer sets may be a number in the range of from 2 to 5. In another embodiment the plurality is 2-3; and still another embodiment the plurality is 3. The concentrations and positions of the primers in a plurality may vary widely. Concentrations of the V region primers may or may not be the same. In one embodiment, the primer closest to the NDN region has a higher concentration than the other primers of the plurality, e.g. to insure that amplicons containing the NDN region are represented in the resulting amplicon. In a particular embodiment where a plurality of three primers is employed, a concentration ratio of 60:20:20 is used. One or more primers (e.g. 1435 and 1437 in FIG. 1D) adjacent to the NDN region (1444) may be used to generate one or more sequence reads (e.g. 1434 and 1436) that overlap the sequence read (1442) generated by J region primer (1432), thereby improving the quality of base calls in overlap region (1440). Sequence reads from the plurality of primers may or may not overlap the adjacent downstream primer binding site and/or adjacent downstream sequence read. In one embodiment, sequence reads proximal to the NDN region (e.g. 1436 and 1438) may be used to identify the particular V region associated with the clonotype. Such a plurality of primers reduces the likelihood of incomplete or failed amplification in case one of the primer binding sites is hypermutated during immunoglobulin development. It also increases the likelihood that diversity introduced by hypermutation of the V region will be capture in a clonotype sequence. A secondary PCR may be performed to prepare the nested amplicons for sequencing, e.g. by amplifying with the P5 (1401) and P7 (1404, 1406, 1408) primers as illustrated to produce amplicons (1420, 1422, and 1424), which may be distributed as single molecules on a solid surface, where they are further amplified by bridge PCR, or like technique.

Base calling in NDN regions (particularly of IgH chains) can be improved by using the codon structure of the flanking J and V regions, as illustrated in FIG. 1E. (As used herein, “codon structure” means the codons of the natural reading frame of segments of TCR or BCR transcripts or genes outside of the NDN regions, e.g. the V region, J region, or the like.) There amplicon (1450), which is an enlarged view of the amplicon of FIG. 1B, is shown along with the relative positions of C read (1442) and adjacent V read (1434) above and the codon structures (1452 and 1454) of V region (1430) and J region (1446), respectively, below. In accordance with this aspect of the invention, after the codon structures (1452 and 1454) are identified by conventional alignment to the V and J reference sequences, bases in NDN region (1456) are called (or identified) one base at a time moving from J region (1446) toward V region (1430) and in the opposite direction from V region (1430) toward J region (1446) using sequence reads (1434) and (1442). Under normal biological conditions, only the recombined TCR or IgH sequences that have in frame codons from the V region through the NDN region and to the J region are expressed as proteins. That is, of the variants generated somatically only ones expressed are those whose J region and V region codon frames are in-frame with one another and remain in-frame through the NDN region. (Here the correct frames of the V and J regions are determined from reference sequences). If an out-of-frame sequence is identified based one or more low quality base calls, the corresponding clonotype is flagged for re-evaluation or as a potential disease-related anomaly. If the sequence identified is in-frame and based on high quality base calls, then there is greater confidence that the corresponding clonotype has been correctly called. Accordingly, in one aspect, the invention includes a method of determining V(D)J-based clonotypes from bidirectional sequence reads comprising the steps of: (a) generating at least one J region sequence read that begins in a J region and extends into an NDN region and at least one V region sequence read that begins in the V regions and extends toward the NDN region such that the J region sequence read and the V region sequence read are overlapping in an overlap region, and the J region and the V region each have a codon structure; (b) determining whether the codon structure of the J region extended into the NDN region is in frame with the codon structure of the V region extended toward the NDN region. In a further embodiment, the step of generating includes generating at least one V region sequence read that begins in the V region and extends through the NDN region to the J region, such that the J region sequence read and the V region sequence read are overlapping in an overlap region.

Somatic Hypermutations. In one embodiment, IgH-based clonotypes that have undergone somatic hypermutation are determined as follows. A somatic mutation is defined as a sequenced base that is different from the corresponding base of a reference sequence (of the relevant segment, usually V, J or C) and that is present in a statistically significant number of reads. In one embodiment, C reads may be used to find somatic mutations with respect to the mapped J segment and likewise V reads for the V segment. Only pieces of the C and V reads are used that are either directly mapped to J or V segments or that are inside the clonotype extension up to the NDN boundary. In this way, the NDN region is avoided and the same ‘sequence information’ is not used for mutation finding that was previously used for clonotype determination (to avoid erroneously classifying as mutations nucleotides that are really just different recombined NDN regions). For each segment type, the mapped segment (major allele) is used as a scaffold and all reads are considered which have mapped to this allele during the read mapping phase. Each position of the reference sequences where at least one read has mapped is analyzed for somatic mutations. In one embodiment, the criteria for accepting a non-reference base as a valid mutation include the following: 1) at least N reads with the given mutation base, 2) at least a given fraction N/M reads (where M is the total number of mapped reads at this base position) and 3) a statistical cut based on the binomial distribution, the average Q score of the N reads at the mutation base as well as the number (M−N) of reads with a non-mutation base. Preferably, the above parameters are selected so that the false discovery rate of mutations per clonotype is less than 1 in 1000, and more preferably, less than 1 in 10000.

### TCRβ Repertoire Analysis

In this example, TCRβ chains are analyzed. The analysis includes amplification, sequencing, and analyzing the TCRβ sequences. One primer is complementary to a common sequence in Cβ1 and Cβ2, and there are 34 V primers capable of amplifying all 48 V segments. Cβ1 or Cβ2 differ from each other at position 10 and 14 from the J/C junction. The primer for Cβ1 and Cβ2 ends at position 16 bp and has no preference for Cβ1 or Cβ2. The 34 V primers are modified from an original set of primers disclosed in Van Dongen et al, U.S. patent publication 2006/0234234, which is incorporated herein by reference. The modified primers are disclosed in Faham et al, U.S. patent publication 2010/0151471, which is also incorporated herein by reference.

The Illumina Genome Analyzer is used to sequence the amplicon produced by the above primers. A two-stage amplification is performed on messenger RNA transcripts (200), as illustrated in FIGS. 2A-2B, the first stage employing the above primers and a second stage to add common primers for bridge amplification and sequencing. As shown in FIG. 2A, a primary PCR is performed using on one side a 20 bp primer (202) whose 3′ end is 16 bases from the J/C junction (204) and which is perfectly complementary to Cβ1 (203) and the two alleles of Cβ2. In the V region (206) of RNA transcripts (200), primer set (212) is provided which contains primer sequences complementary to the different V region sequences (34 in one embodiment). Primers of set (212) also contain a non-complementary tail (214) that produces amplicon (216) having primer binding site (218) specific for P7 primers (220). After a conventional multiplex PCR, amplicon (216) is formed that contains the highly diverse portion of the J(D)V region (206, 208, and 210) of the mRNA transcripts and common primer binding sites (203 and 218) for a secondary amplification to add a sample tag (221) and primers (220 and 222) for cluster formation by bridge PCR. In the secondary PCR, on the same side of the template, a primer (222 in FIG. 2B and referred to herein as “C10-17-P5”) is used that has at its 3′ end the sequence of the 10 bases closest to the J/C junction, followed by 17 bp with the sequence of positions 15-31 from the J/C junction, followed by the P5 sequence (224), which plays a role in cluster formation by bridge PCR in Solexa sequencing. (When the C10-17-P5 primer (222) anneals to the template generated from the first PCR, a 4 bp loop (position 11-14) is created in the template, as the primer hybridizes to the sequence of the 10 bases closest to the J/C junction and bases at positions 15-31 from the J/C junction. The looping of positions 11-14 eliminates differential amplification of templates carrying Cβ1 or Cβ2. Sequencing is then done with a primer complementary to the sequence of the 10 bases closest to the J/C junction and bases at positions 15-31 from the J/C junction (this primer is called C′). C10-17-P5 primer can be HPLC purified in order to ensure that all the amplified material has intact ends that can be efficiently utilized in the cluster formation.)

In FIG. 2A, the length of the overhang on the V primers (212) is preferably 14 bp. The primary PCR is helped with a shorter overhang (214). Alternatively, for the sake of the secondary PCR, the overhang in the V primer is used in the primary PCR as long as possible because the secondary PCR is priming from this sequence. A minimum size of overhang (214) that supports an efficient secondary PCR was investigated. Two series of V primers (for two different V segments) with overhang sizes from 10 to 30 with 2 bp steps were made. Using the appropriate synthetic sequences, the first PCR was performed with each of the primers in the series and gel electrophoresis was performed to show that all amplified.

As illustrated in FIG. 2A, the primary PCR uses 34 different V primers (212) that anneal to V region (206) of RNA templates (200) and contain a common 14 bp overhang on the 5′ tail. The 14 bp is the partial sequence of one of the Illumina sequencing primers (termed the Read 2 primer). The secondary amplification primer (220) on the same side includes P7 sequence, a tag (221), and Read 2 primer sequence (223) (this primer is called Read2_tagX_P7). The P7 sequence is used for cluster formation. Read 2 primer and its complement are used for sequencing the V segment and the tag respectively. A set of 96 of these primers with tags numbered 1 through 96 are created (see below). These primers are HPLC purified in order to ensure that all the amplified material has intact ends that can be efficiently utilized in the cluster formation.

As mentioned above, the second stage primer, C-10-17-P5 (222, FIG. 2B) has interrupted homology to the template generated in the first stage PCR. The efficiency of amplification using this primer has been validated. An alternative primer to C-10-17-P5, termed CsegP5, has perfect homology to the first stage C primer and a 5′ tail carrying P5. The efficiency of using C-10-17-P5 and CsegP5 in amplifying first stage PCR templates was compared by performing real time PCR. In several replicates, it was found that PCR using the C-10-17-P5 primer had little or no difference in efficiency compared with PCR using the CsegP5 primer.

Amplicon (230) resulting from the 2-stage amplification illustrated in FIGS. 2A-2C has the structure typically used with the Illumina sequencer as shown in FIG. 2C. Two primers that anneal to the outmost part of the molecule, Illumina primers P5 and P7 are used for solid phase amplification of the molecule (cluster formation). Three sequence reads are done per molecule. The first read of 100 bp is done with the C′ primer, which has a melting temperature that is appropriate for the Illumina sequencing process. The second read is 6 bp long only and is solely for the purpose of identifying the sample tag. It is generated using a tag primer provided by the manufacturer (Illumina). The final read is the Read 2 primer, also provided by the manufacturer (Illumina). Using this primer, a 100 bp read in the V segment is generated starting with the 1st PCR V primer sequence.

### Isotype Determination

Clonotypes may be constructed from sequence reads of nucleotides encoding immunoglobulin heavy chains (IgHs). In some embodiments, clonotypes typically include a portion of a VDJ encoding region and a portion of its associated constant region (or C region). An isotype is determined from the nucleotide sequence encoding the portion of the C region. In one embodiment, the portion encoding the C region is adjacent to the VDJ encoding region, so that a single contiguous sequence may be amplified by a conventional technique, such as polymerase chain reaction (PCR), such as disclosed in Faham and Willis, U.S. patent publication 2011/0207134, which is incorporated herein by reference. The portion of a clonotype encoding C region is used to identify isotype by the presence of characteristic alleles. In one embodiment between 8 and 100 C-region-encoding nucleotides are included in a clonotype; in another embodiment, between 8 and 20 C-region-encoding nucleotides are included in a clonotype. In one embodiment, such C-region encoding portions are captured during amplification of IgH-encoding sequences as described more fully below. In such amplifications, one or more C-region primers are positioned so that a number of C-region encoding nucleotides in the above ranges are captured in the resulting amplicons.

There are five types of mammalian Ig heavy chain denoted by the Greek letters: α, δ, ε, γ, and μ. The type of heavy chain present defines the class of antibody; these chains are found in IgA, IgD, IgE, IgG, and IgM antibodies, respectively. Distinct heavy chains differ in size and composition; α and γ contain approximately 450 amino acids, while μ and ε have approximately 550 amino acids. Each heavy chain has two regions, the constant region and the variable region. The constant region is identical in all antibodies of the same isotype, but differs in antibodies of different isotypes. Heavy chains γ, α and δ have a constant region composed of three tandem (in a line) Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain. Nucleotide sequences of human (and other) IgH C regions may be obtained from publicly available databases, such as the International Immunogenetics Information System (IMGT) at http://www.imgt.org.

### Example

Samples of 6 patients suffering from psoriatic arthritis were analyzed in accordance with the invention. Three patients had a single time point, and the other patients had 2-3 time points. For each time point there were two samples: from Peripheral Blood Mononuclear Cells (PBMC) and Synovial Fluid (SF). For each sample RNA was prepared, cDNA generated, IgH and TCRB genes amplified, after which the amplification products were subjected to deep sequencing (100K-800K reads for each sample) and frequencies of the different clonotypes in each sample were determined. The high data quality was validated by the high frequency of mapping, low measured error rate, high clonotype in frame frequency, and high repeatability of clonotype frequencies in duplicate samples. There was significant correlation in TCRB clonotype frequencies between different samples of the same patient with higher correlation between different time points of the same tissue compared to different tissues at the same time point. The clonotype frequency distribution in SF samples was substantially shifted towards higher frequency range compared to that observed in PBMC. Correlation of IgH clonotype frequency in different samples of the same patient was lower than seen in TCRB, and it was greatest in SF-SF comparison. There were multiple features in the IgH repertoire that distinguish SF from PBMC samples. These include total IgH molecules per 1 ug of RNA, IgG fraction, IgD fraction, Diversity in IgM class and IgD class, and mutation per IgM clonotype. Some of these features distinguish some of the patients from each other. Particularly patients 001, 002, 004, and 006 generally have similar values for these measures. Patients 003 and 005 differ from the rest of the patients in some of these measures. In addition patient 3 clonotype frequencies has substantially less correlation between the two SF samples compared with correlation observed in clonotype frequencies in SF samples of patients 1 and 2 (no serial time points for the rest of the patients). Having identified patients 003 and 005 as distinct from the rest of the patients it is noted that these patients were not being actively treated with a DMARD, while patients 001, 002, and 006 were on a DMARD (patient 004 is unknown).

Psoriatic Arthritis (PsA) is one of the seronegative spondyloarthropathies that is associated with HLA-B27. A variety of drugs including TNF blockers are used and assessment of response is generally done by clinical symptoms (joint counting). Biomarkers like ESR and CRP are used but their performance (sensitivity and specificity) is less than optimal. Assessment of levels of PsA-specific T and/or B cells may ultimately be a good method to determine disease activity with high sensitivity and specificity. The T-cell and B-cell repertoire profiles in blood and synovial fluids in 6 PsA patients were studied. For most of the time points/tissues, two samples were prepared; one that was used for RNA preparation and the other was stored as cells. In all three patients with serial samples, steroid injection in the knee joint and synovial fluid aspiration was done.

Briefly, the assay of each sample includes two stages of PCR with the first PCR using multiplex primers, and the second using one pair of primer sequences, as described above. For TCRB (FIGS. 2A-2C), a set of primers complementary to the different V segments with a single primer complementary to the C segment was used. For IgH (FIG. 1A-1E), three sets of V primers were designed along with one set of C segment primers, as described above. Three reactions are performed using the C primers and one of the three V primer sets. The primer sets were designed in such a way that each V segment sequence is amplifiable in each of the three reactions greatly increasing the likelihood that sequences with somatic hypermutations can still be amplified in at least one pool. The amplification was optimized to ensure small bias (<2 fold of any sequence to the average) in the amplification of the different sequences. After the first stage is completed, the three reactions are mixed and a second stage amplification is performed to append the sequences that will be later utilized for the priming of sequencing. The second stage amplification also allows the incorporation of a sample-specific index sequences. An Illumina HiSeq platform was used to obtain the sequence information. Individual amplified molecules are separated on a solid surface on which they are copied using solid phase amplification to generate clusters with as many as 1,000 copies of the molecule in question. These clonal “clusters” are then sequenced base by base in a sequencing-by-synthesis approach. Using a conventional manufacturer's protocol, three sequencing reactions are performed on each cluster: 100 bp from each of the two directions and a further 6 bp read in the forward direction using a complement to sequencing primer for the index sequence. Typically >100K sequences per sample consisting of sequencing reads from all three frames are obtained. In general more sequencing reads are collected than the number of B cells in order to ensure that maximum use is made of the biological material. As shown in FIG. 1B, for the IgH assay, each sequence is amplified in each of the three reactions generating three products with different sizes. As above, when the products are sequenced three reads using three primers are obtained. One of the reads is a short read (6 bp) to sequence the index to determine the sample to which the molecule belongs. The other two reactions are 100 bp each obtained from both directions of the sequence (the J side and the V side). The 100 bp read from the J side (J read) typically covers the J and D segments as well as a portion of the V segment. Therefore information from the J read encompasses the most unique sequence, the CDR3. The V read is totally contained within the V segment for the larger of the two products allowing for the appropriate mapping of the V segment. The V and J reads in the smallest of the three products overlap allowing for the correction of errors in the J read, particularly at the end of the read where the sequencing

For three patients (patients 004, 005, and 006) there was only one time point. For 2 patients (patients 001 and 003) there were two time points, and for one patient (patient 002) there were three time points. For each of the time points there were samples from PBMC and SF. For each of these 20 samples (except patient 1) two tubes was provided for each sample. One of the tubes from each sample has been stored in DMSO and was not utilized. The other tube stored in RNAProtect was used for RNA preparation using the AllPrep kit (Qiagen). Each of the prepared RNA samples was quantitated using Quant iT to assess the total amount of RNA prepared. One sample (Dubs001_PBMC_{—}10_{—}3_{—}10_DMSO) had very little obtained RNA. As much as 2 μg of RNA of each sample was used to synthesize cDNA that was in turn used to assess the total TCRB and IgH molecules by real time PCR. The same cDNA was then used to amplify TCRB and IgH that were later sequenced.

Samples QC included measurement of total RNA yield (Table 1) as well total TCRB and IgH molecule in the cDNA reaction by real time PCR. The obtained data are shown in Tables 1 and 2. One sample (Dubs001_PBMC_{—}10_{—}3_{—}10_DMSO) has significantly lower RNA yield than the rest.

TABLE 1
Sample quantitation

| | | RNA amount used to |
|---|---|---|
| Sample name | RNA total yield (ng) | synthesize cDNA (ng) |
| Dubs001_SFMC_10_3_10_DMSO | 1,779 | 830 |
| Dubs001_SFMC_25_5_10_PMCC | 7,904 | 2,000 |
| Dubs002_SFMC_1_6_10 | 4,510 | 2,000 |
| Dubs002_SFMC_6_7_10 | 5,808 | 2,000 |
| Dubs002_SFMC_14_9_10 | 15,258 | 2,000 |
| Dubs003_SFMC_22_6_10 | 8,325 | 2,000 |
| Dubs003_SFMC_24_8_10 | 5,163 | 2,000 |
| Dubs004_SFMC_26_10_10 | 6,733 | 2,000 |
| Dubs005_SFMC_17_11_10 | 5,873 | 2,000 |
| Dubs006_SFMC_2_12_10 | 10,336 | 2,000 |
| Dubs001_PBMC_10_3_10_DMSO | 137 | 64 |
| Dubs001_PBMC_25_5_10_PMCC | 3,593 | 1,677 |
| Dubs002_PBMC_1_6_10 | 4,173 | 1,947 |
| Dubs002_PBMC_6_7_10 | 9,356 | 2,000 |
| Dubs002_PBMC_14_9_10 | 4,611 | 2,000 |
| Dubs003_PBMC_22_6_10 | 7,306 | 2,000 |
| Dubs003_PBMC_24_8_10 | 2,338 | 1,091 |
| Dubs004_PBMC_26_10_10 | 4,054 | 1,892 |
| Dubs005_PBMC_17_11_10 | 6,469 | 2,000 |
| Dubs006_PBMC_2_12_10 | 6,194 | 2,000 |

The RNA yield and the amount used to synthesize cDNA is shown for all 20 used samples. The names of the samples are generated by concatenating the patient name (Dub001-Dub006), the type of sample (SF or PBMC), the date of collection, and when the sample was not stored in RNAProtect that information was added.

As can be seen in table 2 the total IgH in the cDNA for SF samples is generally higher than in PBMC. In addition this gross measure in SF samples can classify the patients into two groups separated by ˜30 fold of IgH amount. Patient 3 and 5 have <500K IgH molecules (per 2 μg of starting RNA) and patients 1, 2, and 4 have >17M IgH molecules (per 2 μg of starting RNA). IgH amount in PBMC samples and patient 6 generally fell in between the above two groups.

TABLE 2
Number of TCRB and IgH molecules that were in the cDNA and the amplification

| | Total TCRB | TCRB molecules | Total IgH | IgH molecules |
|---|---|---|---|---|
| | molecules in | input into PCR | molecules in | input into PCR |
| Sample_name | cDNA | reaction | cDNA | reaction |
| Dubs001_SFMC_10_3_10_DMSO | 5,045,378 | 2,018,151 | 135,764,502 | 61,094,026 |
| Dubs001_SFMC_25_5_10_PMCC | 6,611,431 | 2,644,572 | 17,814,283 | 8,016,427 |
| Dubs002_SFMC_1_6_10 | 8,723,835 | 3,489,534 | 54,378,426 | 24,470,292 |
| Dubs002_SFMC_6_7_10 | 3,494,201 | 1,397,680 | 57,878,776 | 26,045,449 |
| Dubs002_SFMC_14_9_10 | 1,585,527 | 634,211 | 46,687,437 | 21,009,347 |
| Dubs003_SFMC_22_6_10 | 1,885,520 | 754,208 | 292,187 | 131,484 |
| Dubs003_SFMC_24_8_10 | 4,484,548 | 1,793,819 | 430,762 | 193,843 |
| Dubs004_SFMC_26_10_10 | 13,407,446 | 5,362,978 | 55,521,033 | 24,984,465 |
| Dubs005_SFMC_17_11_10 | 8,603,731 | 3,441,493 | 213,893 | 96,252 |
| Dubs006_SFMC_2_12_10 | 8,969,095 | 3,587,638 | 3,446,095 | 1,550,743 |
| Dubs001_PBMC_10_3_10_DMSO | 165,507 | 66,203 | 364,746 | 164,136 |
| Dubs001_PBMC_25_5_10_PMCC | 5,598,198 | 2,239,279 | 8,723,835 | 3,925,726 |
| Dubs002_PBMC_1_6_10 | 7,285,169 | 2,914,068 | 24,846,358 | 11,180,861 |
| Dubs002_PBMC_6_7_10 | 2,403,210 | 961,284 | 11,753,044 | 5,288,870 |
| Dubs002_PBMC_14_9_10 | 3,063,036 | 1,225,215 | 6,083,757 | 2,737,691 |
| Dubs003_PBMC_22_6_10 | 8,544,301 | 3,417,720 | 8,723,835 | 3,925,726 |
| Dubs003_PBMC_24_8_10 | 2,020,850 | 808,340 | 2,257,870 | 1,016,042 |
| Dubs004_PBMC_26_10_10 | 5,676,346 | 2,270,538 | 10,890,230 | 4,900,603 |
| Dubs005_PBMC_17_11_10 | 3,986,057 | 1,594,423 | 3,877,058 | 1,744,676 |
| Dubs006_PBMC_2_12_10 | 6,611,431 | 2,644,572 | 7,647,364 | 3,441,314 |

(1)

Only a fraction of the cDNA synthesis sample is used in the amplification of each of TCRB and IgH. Described here is the total number of TCRB and IgH molecules in the cDNA and in the amplification reaction that is later subjected to sequencing.

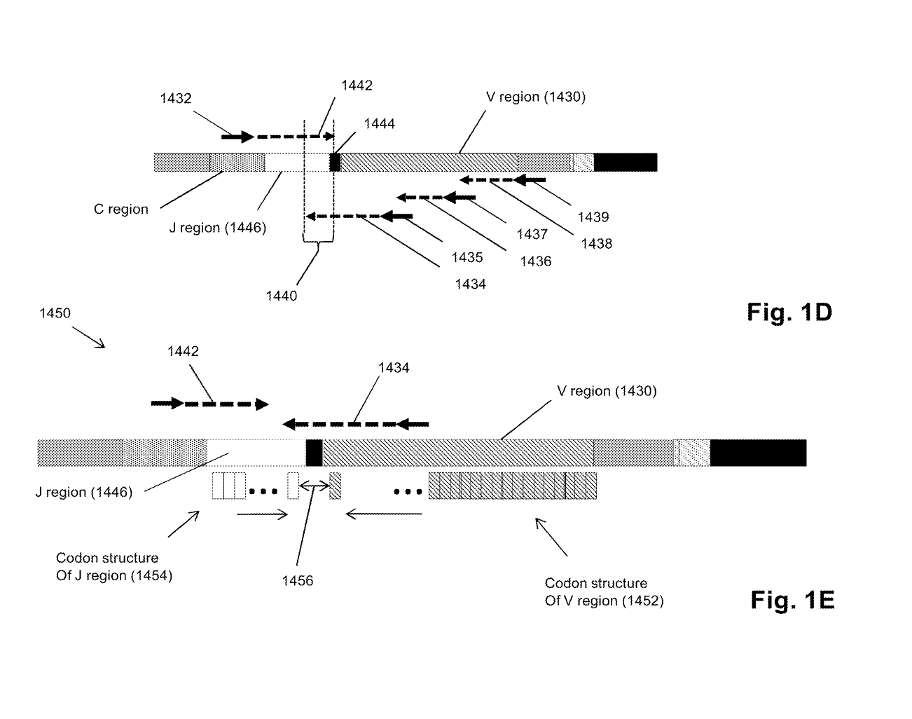

To initially investigate the quality of the data, information from the mapping was used. For a read to map, the beginning of its read 1 (forward direction) needs to map to a J segment, read 2 (reverse direction) needs to map to a V segment, and the end of read 1 needs to map to a V segment. The frequency of the reads that map to these different segments is high (FIG. 3). In addition we can look at the mapping per base error rate. The error rate is generally low and consistent with the position effect in the read, which validates the high quality of the data.

In FIG. 3, the X axis is the error rate for sequences that map to a specific segment, and the Y axis is the frequency of all sequences that map to the specific type of segment. Each square is for one experiment. Each experiment has three squares: for the beginning of read 1 mapping J segment (300), V2 (302) (read 2 mapping to V segment), and V1 (304) (end of read 1 mapping to V segment). The error rate for each of the 3 segments is predictable from the position of the segment in the read. For example the section mapping to the J segment (300) is in the beginning of read1 and has significantly less error rate than V1 (304) mapping which is at the end of the same read. Solid squares show SF samples and open squares are for PBMC samples. For most samples (except for Dubs001_PBMC_{—}10_{—}3_{—}10_DMSO which had significantly lower number of input molecules) there were ˜100K-700K mapped (ie, mapped to a J segment, V1, and V2) reads.

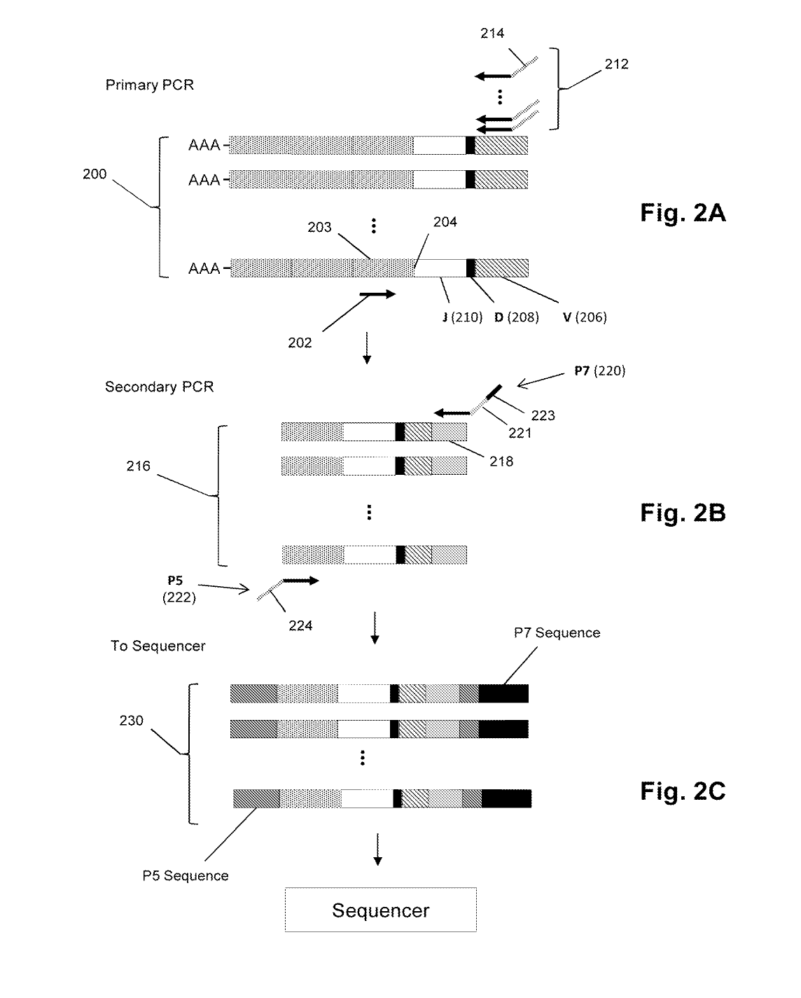

FIG. 4 shows total TCRB mapped reads. The total mapped read count (Y axis) is shown for the different experiments (X axis). Solid squares show SF samples and open squares show PBMC samples. As anticipated Dubs001_PBMC_{—}10_{—}3_{—}10_DMSO (400) had very few reads given the much lower input molecules. Mapped sequences were then put into clonotypes. A clonotype requires that at least two exact matches are present for a particular sequence as singletons are eliminated. The frequency of cloning then is inversely correlated with the fraction of mapped reads that are singleton. If the sequencing depth is very high then each initial cell will have many reads representing it and hence the fraction of singleton reads will be small (and by extension the frequency of cloning will be high). In general in this experiment the depth of sequencing (number of reads) we have done is less than the number of input TCRB RNA molecules (table 2). However, for each of the T cells usually there are ˜10 TCRB RNA molecules (our unpublished observations), and therefore for most samples we probably covered most of the input cells.

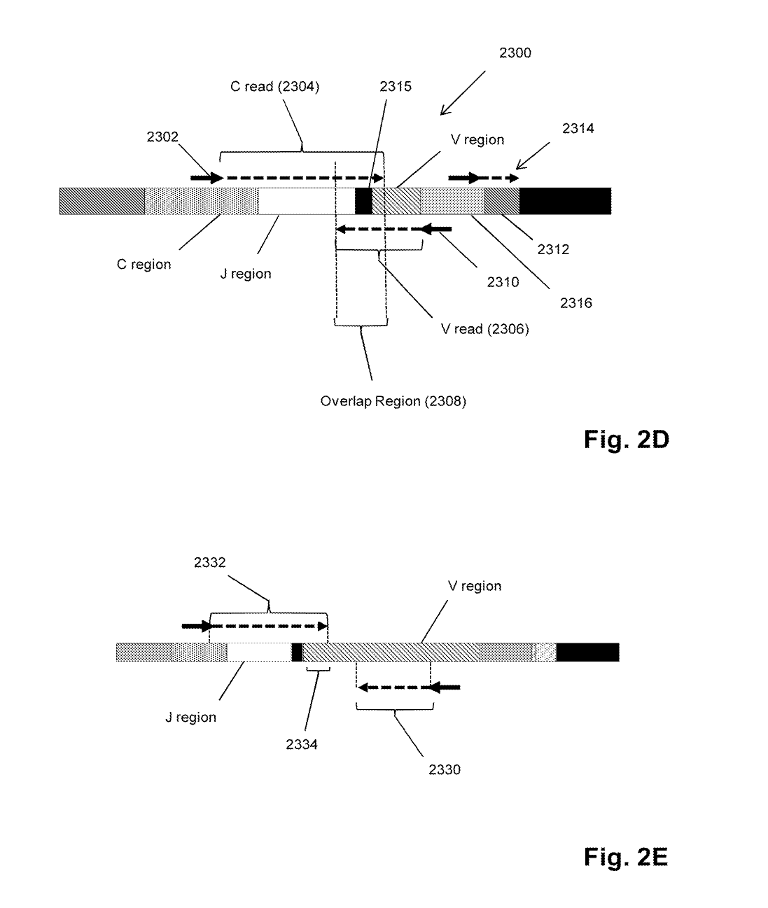

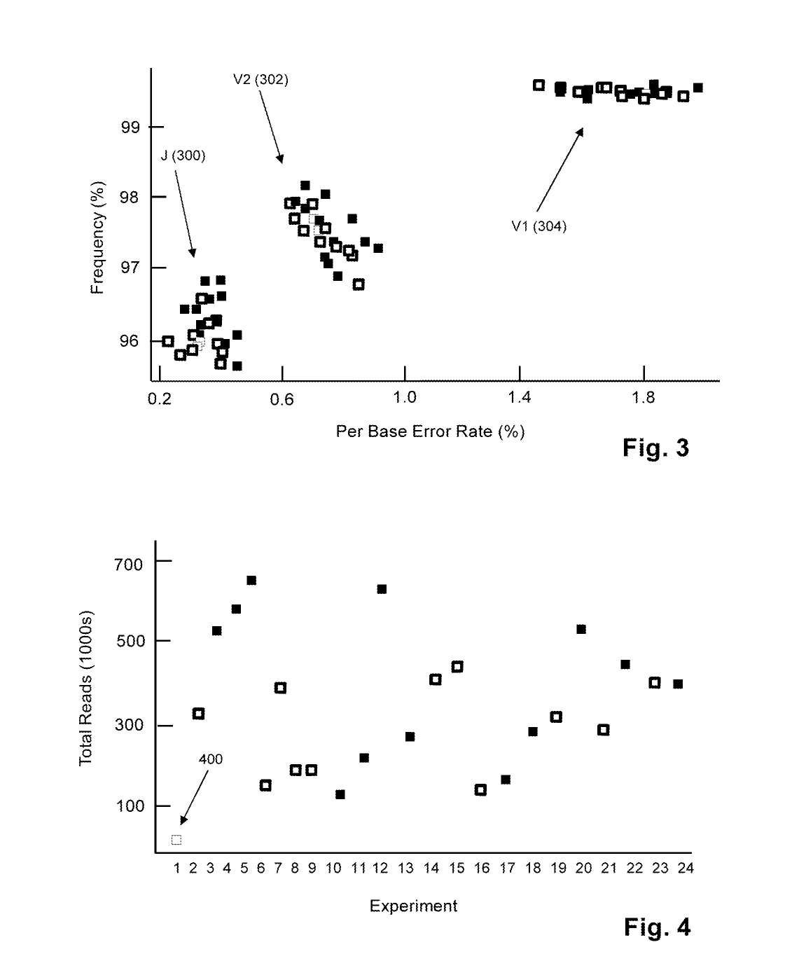

FIG. 5 shows that the frequency of cloning for the SF samples is higher than that for PBMC. This is not due to lower starting material (table 2) but is likely reflecting lower diversity for these samples as will be discussed later. The total cloned reads counts (Y axis) is calculated as the total mapped reads multiplied by the cloning frequency (X axis). Solid squares show SF samples and open squares show PBMC samples. To validate the quality of the data and the accuracy of the algorithms we assessed the fraction of cloned reads that are in frame. As shown in FIG. 6, on average ˜99% of cloned reads are in frame. This validates that we are assembling valid clonotypes. The 1% is a ceiling on the error we are generating as some of these reads might be due to some leaky expression of the excluded alleles.

FIG. 6 shows the fraction of TCRB sequences that are in frame. The X axis shows the different experiments and the Y axis depicts the fractions of all cloned reads that are in frame. Very high in frame values are obtained (˜99%) confirming the high quality of the full pipeline from the amplification to the algorithm.

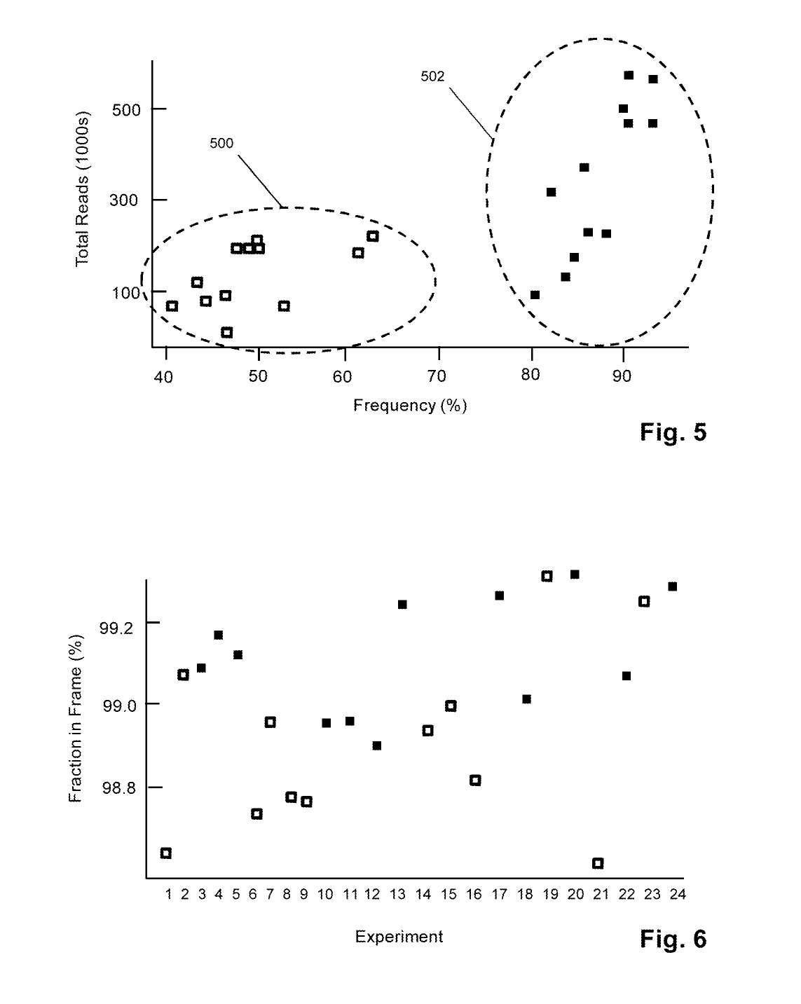

To assess the replication of the data we have done the assay for several samples in duplicates. The duplicates were separated at the point of cDNA synthesis. Two examples of duplicates are shown in FIGS. 7A-7B demonstrating the high degree of replication. Amplification and sequencing were done in duplicates for several samples. Two examples are shown. FIG. 7A) −log₁₀ of the frequency of each clonotype in duplicate runs of sample SFMC_{—}25_{—}5_{—}10_PMCC is shown. FIG. 7B) −log₁₀ of the frequency of each clonotype in duplicate runs of sample Dubs003_PBMC_{—}22_{—}6_{—}10 is shown. Overlap (at the nucleotide) level of samples from different individuals have minimal overlap (generally <20 clonotypes). Obviously the deeper the sequencing performed, the higher the number of clonotypes shared across individuals.

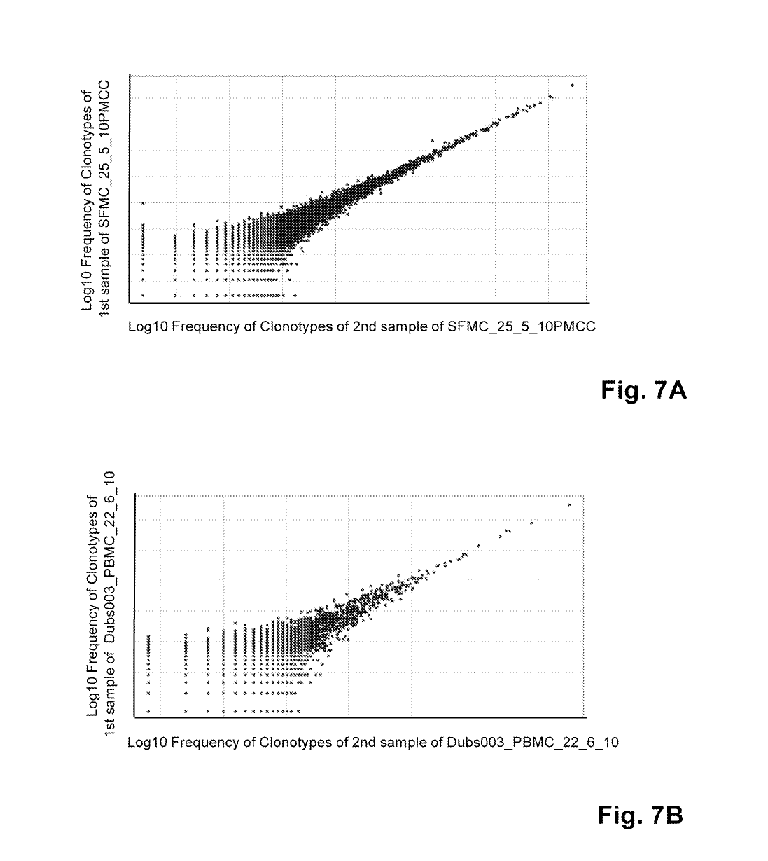

Clonotypes within an individual tend to be more conserved in the same tissue between different time points compared to the same time point different tissues. In other words there is a certain consistency to the clonotypes and their frequency in SF (or PBMC) in time points that are 1-2 months apart. There is also significant overlap between the clonotypes found in SF and PBMC at the same time point but to a lower extent compared to what is seen SF (or PBMC) across time points. FIGS. 8A-8C show typical examples of SF-SF; PBMC-PBMC; and SF-PBMC clonotype comparison in the same patient. The patients clinical scores (tender or swollen joint scores) did not seem to differ substantially in the various time points. Therefore we could not search for features that correlate with disease activity.

FIGS. 8A-8C show log₁₀ of the clonotype frequencies observed in different samples of the same patient. Each dot is for a specific clonotype sequence. FIG. 8A shows a comparison of two SF samples that are 1 month apart. FIG. 8B shows comparison of two PBMC samples that are also 1 month apart. FIG. 8C shows the comparison between PBMC (X axis) and SF (Y axis) at the same time point. The clonotype frequency distribution in SF samples is significantly different from that of PBMC samples. This is suggested by the different cloning frequency discussed above (FIG. 3). FIGS. 8A-8C show a dramatic difference in the clonotype frequency distribution between SF and PBMC samples with the SF clonotype distribution shifted to the right (towards higher frequency clonotypes). The frequency of the top clonotypes in SF is generally observed to be at the 1-7% range. But this is does not explain the frequency distribution shift. There are many clones that are observed at higher frequency in the SF than blood. For example there are on average 94+/−15 clonotypes in SF and 10+/−4 in a PBMC sample observed at a frequency of 10⁻³ or higher.

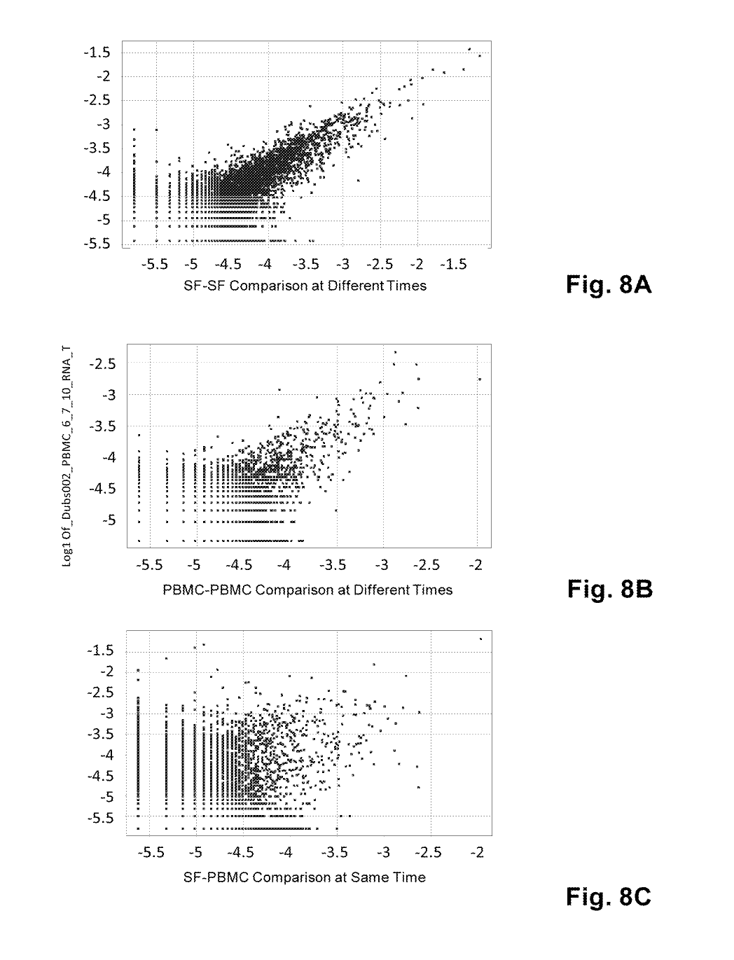

FIGS. 9A-9B show TCRB clonotype frequency distribution in SF and PBMC samples. Log₁₀ clonotype frequencies are binned in the X axis and the sum of the clonotype frequencies in each bin is shown on the Y axis. The absolute scale on the Y axis adds to much more than 100% and is not the focus. Instead the focus in the relative height of the bars in the different bins. For example in FIG. 9A) where data from PBMC are shown, the bins with the most reads are those that represent low frequency clonotypes (˜−log₁₀ of −5). On the other hand in FIG. 9B) where data from SF are shown the bins have more equivalent number of reads with many of the reads observed in bins with −log₁₀ of ˜−4. Very high frequency clonotypes in the SF might be involved in the pathogenesis of PsA. Public clonotypes common among the patients would support that these clonotypes are relevant to PsA. Differing HLA haplotypes among the patients would decrease the likelihood of identifying a common sequence among patients. We have not identified a common clonotype sequence present at high frequency among the 6 patients. We will be performing more detailed analysis to consider the possibility of the presence of related sequences or motifs among the patients.

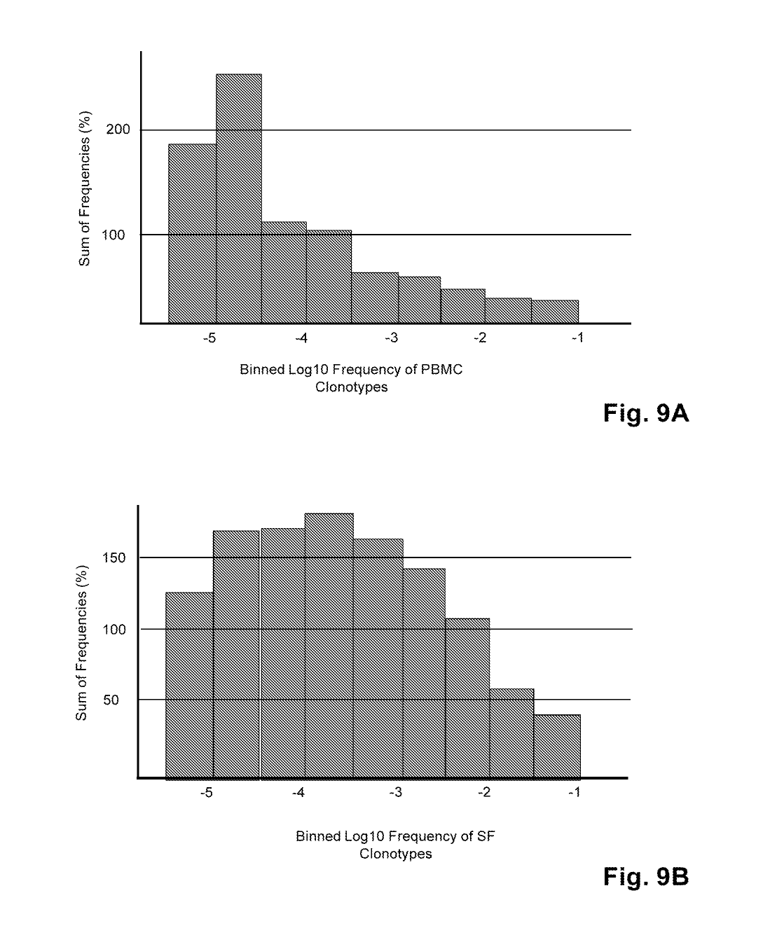

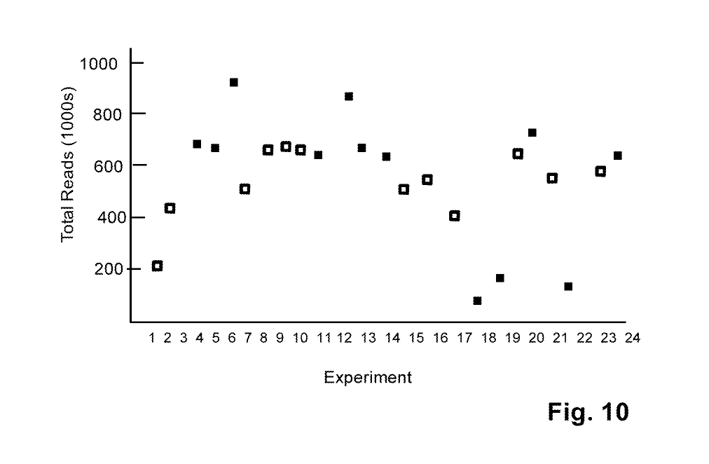

The obtained sequencing reads were mapped to J and V segments. We obtained ˜200,000-800,000 mapped reads for the different samples (FIG. 10). The total mapped read count (Y axis) is shown for the different experiments (X axis). Solid squares show SF samples and open squares are for PBMC samples. The mapped reads were then put into clones with singleton reads eliminated. The frequency of cloning is then the fraction of mapped reads that are have at least one other exact match. The frequency of cloning in SF samples is much higher than that for PBMC (FIG. 11) indicating there is less diversity in SF samples as we will demonstrate in more details below. The total cloned read counts (Y axis) are calculated as the total mapped reads multiplied by the cloning frequency (X axis). Solid squares show SF samples and open squares show PBMC samples. The cloning frequency of all the SF samples is >95% whereas the median for the PBMC samples is ˜80%.

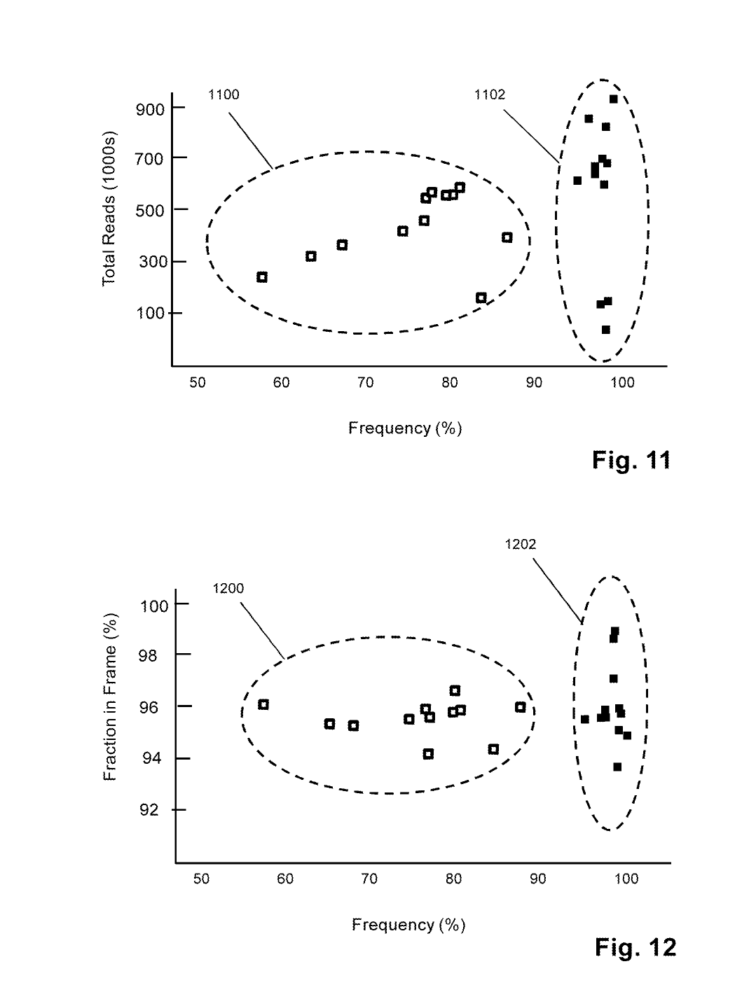

To validate the quality of the data we assessed the fraction of sequences that generate an in frame IgH molecule. We noted that irrespective of the cloning frequency the in frame frequency is ˜96% (FIG. 12). This validates the reliability of the used amplification, sequencing, and algorithms. FIG. 12 shows the fraction of IgH sequences that are in frame. The X axis shows the cloning frequency of the different experiments and the Y axis depicts the fractions of all cloned reads that are in frame. Very high in frame values are obtained (˜96%) confirming the high quality of the full pipeline from the amplification to the algorithm. Solid squares show SF samples and open squares are for PBMC samples.

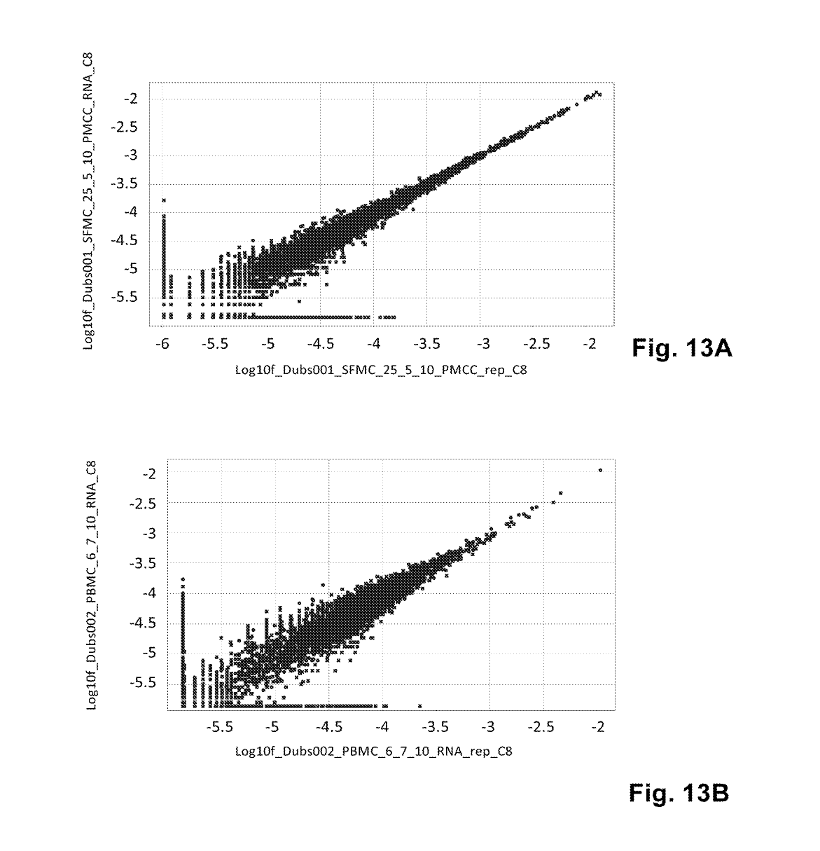

To validate the reliability of the data we ran duplicate samples (independent amplifications) of a SF sample and another of a PBMC sample. In both cases the clonotype profiles have high degree of replication (FIG. 13). Amplification and sequencing were done in duplicates for several samples. Two examples are shown. In FIG. 13A) −log₁₀ of the frequency of each clonotype in duplicate runs of sample SFMC_{—}25_{—}5_{—}10_PMCC is shown. In FIG. 13B) −log₁₀ of the frequency of each clonotype in duplicate runs of sample Dubs002_PBMC_{—}6_{—}7_{—}10 is shown. Overlap (at the nucleotide) level of samples from different individuals have minimal overlap (generally <20 clonotypes). Obviously the deeper the sequencing performed, the higher the number of clonotypes shared across individuals.

The clonotypes across time points and across tissues and across time are significantly more variable compared to TCRB (data not shown). In general the 2 months correlation (or clonotype overlap) between SF-SF samples is better than PBMC-PBMC samples. The latter is often about the same or higher than correlation observed between PBMC and SF at the same time point (data not shown).

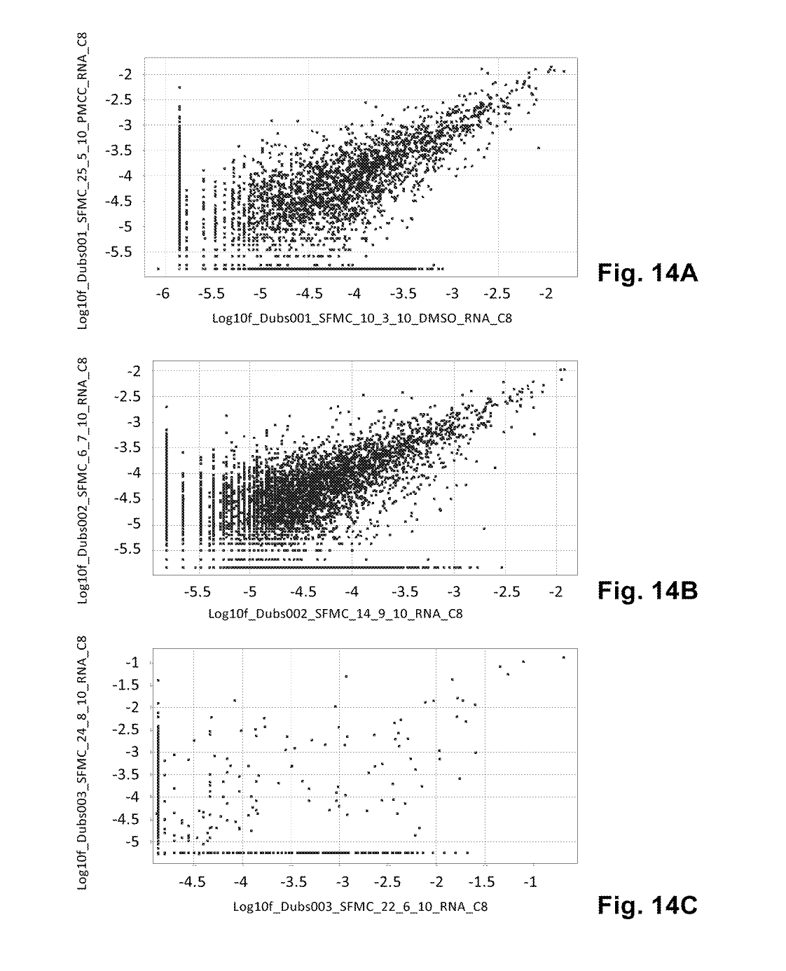

The extent of clonotype overlap between samples of the same patients differs between patients. For example we found some differences in the clonotype overlap between SF-SF across two month comparison in the 3 available patients. FIG. 14 shows the SF clonotype comparison in the three patients with serial time points. As can be seen in FIG. 14 the clonotype overlap between the two time points is dramatically lower in patient 3 compared to the other two patients. The difference is quantitated in table 3.

FIGS. 14A-14C show IgH clonotype comparison in the same patient at different time points, as log₁₀ of the clonotype frequencies observed in SF of the same patients at time points ˜2 months apart. Each dot is for a specific clonotype sequence. Patients 001, 002, and 003 are shown in FIGS. 14A, 14B, and 14C, respectively. We computed the extent of clonotypes overlap in two different samples. We basically asked whether clonotypes observed in one sample are also present in the other sample. Because of concerns regarding Poisson statistics, we only asked whether clonotypes present at 10⁻⁴ (−log 10 value of −4, plotted as −4 in FIG. 14) or higher level in one sample are present in the other. We scored as positives (overlapping) clonotypes present at a limit ˜3 fold lower (−log 10 value of −4.5, plotted as −4.5 in FIG. 14) in the other samples. We considered the negatives (absent) as those which are absent (on the axis in FIG. 14) in the other sample. We did the symmetrical inquiry to assess whether clonotypes present in the second sample at −log 10 value of −4 or higher are present at −log 10 value of −4.5 or higher (overlapping) or absent in the first sample. The sum of the overlapping and absent clonotypes in the two comparisons is reported.

TABLE 3
Extent of IgH clonotype overlap between
same patient SF samples 2 months apart

| | Total overlapping | Total absent | Overlapping/total |
|---|---|---|---|
| Patient name | clonotypes | clonotypes | clonotypes |
| 001 | 1304 | 516 | 0.72 |
| 002 | 1381 | 488 | 0.74 |
| 003 | 88 | 594 | 0.13 |

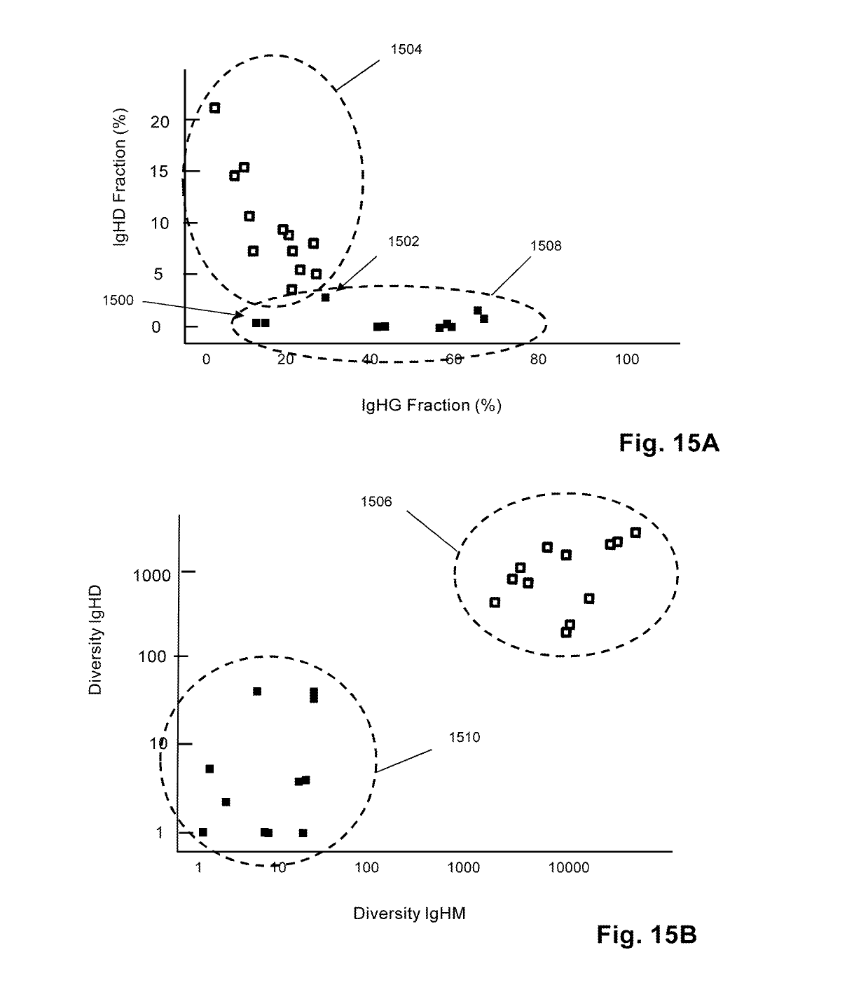

Features distinguishing SF from PBMC samples. There were many features of the SF samples that were distinct from PBMC samples. SF samples tended to have more IgG and less IgD reads (FIG. 15A). In addition SF clonotypes tended to be less diverse. This is particularly true when we limited ourselves to clonotypes that were predominantly IgM or IgD. This can be seen by looking at clonotype distribution as we showed for TCRB sequences. We have wanted to reduce the diversity to a metric of a single number, although many other measures of diversity may be used in accordance with the invention, e.g. as disclosed in Peet, Ann. Rev. Ecol. Systematics, 5: 285-307 (1974); Hill et al, FEMS Microbiol. Ecol., 43: 1-11 (2003); Pielou, Introduction to Mathematical Ecology (Wiley-Interscience, New York, 1969); and like references. We have calculated the number of clonotypes that are in the top 10% or 25% of the reads. The less clonotypes that would account for 25% of reads the less diversity the sample has. We can compute the top 10% or 25% of reads among all the clonotypes. In addition we can compute that for only specific classes of clonotypes. As can be seen in FIG. 15B, the diversity of IgM and IgD clonotypes is remarkably restricted in SF compared to PBMC samples. Finally we have also noted that in SF samples IgM clonotypes in the top 25% of reads tended to have more mutations per clonotypes than in PBMC (table 4). This observation may be partly explained by the fact that there is less diversity of IgM in SF and hence these clonotypes have higher frequency which tends to have higher mutation rates.

Different parameters are calculated on each sample. Each sample is represented by one dot. Solid squares show SF samples and open squares are for PBMC samples. FIG. 15A) shows the IgG fraction vs. IgD fraction. The fraction is the proportion of all mapped reads that are IgG or IgD. There is a complete separation between PBMC and SF samples in IgD fraction and a difference in IgG fraction. The lowest SF IgG fraction is for patients 003 (1500) and 005 (1502). Difference between SF and PBMC in the clonotype diversity within IgM and IgD classes is shown in FIG. 15B. The IgM measure (shown on the X axis) is the number of clones that are in the top 25% of IgM reads. Similarly the IgD diversity measure (shown on the Y axis) is the number of clones in the top 10% of IgD reads (the results are generally similar using the top 25% of IgD). The smaller number of clonotypes in the top fixed proportion of reads indicate that there those clonotypes have higher frequency and is reflective of lower diversity in the sample. Both the IgM and IgD diversity measures clearly distinguish PBMC from SF samples. Patient 005 (and one replicate samples of patient 001) have the lowest IgD diversity (highest number of clones in the top 10% of reads).

Stratifying SF samples in different types. The above features clearly distinguish SF from PBMC samples. We looked to assess whether some of these features distinguish different SF samples from each other. In table 4 we list the values of the various factors that are different between SF and PBMC in the different samples. The IgM25 clone count clearly distinguishes PBMC (range 1.3K-18.8K) from SF samples (range 1-13). Mutations per clone in these clonotypes in PBMC is relatively low (0.9-3.7 mutations per clone) if we exclude sample 001_PBBMC_{—}10_{—}3_{—}10 given its low level of RNA yield. The mutation per clone in the SF samples of standard PsA group is higher (range 4.5-17.8 mutations per clone). We have noted that IgG fraction is generally higher in SF than PBMC except for patients 003 and 005 with IgG fraction in SF of patient number 003 (12.9%) in the middle of the range of values (3.2%-24.7%) obtained in PBMC. IgG fraction in SF in patient 005 is slightly higher (29.4%) than obtained for the PBMC samples but lower than what is observed in the rest of the SF samples (range 41.9%-63.9%). IgG fraction in SF samples show different range of values for patients 001, 002, 004, 006 (will call standard PsA group) from patients 003 and 005 Of note these two patients (003 and 005) also stand out as the SF samples with the lowest IgH molecule per μg of RNA. The range of IgH molecules per 1 μg of RNA for these patients is 107K-215K, and the range for the rest of SF samples is (1.7M-163M). Similarly all the SF samples from the standard PsA group have low IgD fraction (0.04%-1.18%). Patient 003 has similar levels but patient 005 has the highest value (2.95%) in SF samples. Finally measure of IgD diversity, IgD10 clone count, shows that all SF samples have significantly less diversity (1-32 clones in the top 10% of reads) than PBMC samples (273-3,356 clones). Within the SF samples all samples except patient 005 and one sample of 001 the number of clones in the top 10% of reads is less than 5 indicating the dominance of a high frequency clone. This analysis clearly distinguishes SF and PBMC. It may also stratify the patients into a standard PsA group (patients 001, 002, 004, and 006) that has very high number of IgH molecule per 1 μg RNA, high IgG fraction, low IgD fraction, low diversity in the IgD and IgM clones, and high mutations per clone in the IgM clonotypes in the top 25% of reads. Patients 003 and 005 share some of the same pattern as the standard PsA group, but differ in some features.

TABLE 4
Different IgH parameters in PBMC and SF samples.

| | | IGHD | IGHG | IGHD10 | IGHM25 | IGHM 25 |
|---|---|---|---|---|---|---|
| | IgM molecules | Fraction | Fraction | Clone | Clone | Mutations |
| Experiment Name | per 1 μg RNA | (%) | (%) | Count | Count | per Clone |
| Dubs001_PBMC_10_3_10 | 5,699,286 | 7.78 | 9.5 | 306 | 1,337 | 5.8 |
| Dubs001_PBMC_25_5_10 | 5,202,559 | 20.88 | 3.2 | 3,356 | 18,788 | 0.9 |
| Dubs002_PBMC_1_6_10 | 12,758,280 | 3.95 | 19.9 | 1,169 | 2,418 | 3.7 |
| Dubs002_PBMC_6_7_10 | 5,876,522 | 5.96 | 21.8 | 334 | 6,963 | 2.4 |
| Dubs002_PBMC_14_9_10 | 3,041,878 | 9.34 | 17.0 | 655 | 8,732 | 1.9 |
| Dubs003_PBMC_22_6_10 | 4,361,918 | 14.75 | 5.3 | 2,369 | 13,849 | 1.8 |
| Dubs003_PBMC_22_6_10_rep | 4,361,918 | 15.44 | 6.8 | 2,438 | 13,197 | 1.6 |
| Dubs003_PBMC_24_8_10 | 2,068,997 | 11.07 | 8.4 | 829 | 1,969 | 3.3 |
| Dubs004_PBMC_26_10_10 | 5,756,830 | 8.91 | 24.7 | 849 | 3,339 | 2.4 |
| Dubs005_PBMC_17_11_10 | 1,938,529 | 9.73 | 15.6 | 1,516 | 7,696 | 1.9 |
| Dubs006_PBMC_2_12_10 | 3,823,682 | 7.87 | 18.0 | 1,932 | 5,748 | 3.0 |
| Dubs002_PBMC_6_7_10_rep | 5,876,522 | 5.21 | 24.8 | 273 | 6,455 | 2.1 |
| Dubs001_SFMC_10_3_10 | 163,577,770 | 0.04 | 41.1 | 3 | 13 | 9.7 |
| Dubs001_SFMC_25_5_10 | 8,907,141 | 0.05 | 42.6 | 26 | 13 | 8.9 |
| Dubs002_SFMC_1_6_10 | 27,189,213 | 0.28 | 57.2 | 1 | 13 | 10.5 |
| Dubs002_SFMC_6_7_10 | 28,939,388 | 0.29 | 55.0 | 1 | 8 | 10.6 |
| Dubs002_SFMC_14_9_10 | 23,343,719 | 0.12 | 56.8 | 1 | 7 | 9.9 |
| Dubs003_SFMC_22_6_10 | 146,093 | 0.35 | 12.9 | 1 | 1 | 10 |
| Dubs003_SFMC_24_8_10 | 215,381 | 0.38 | 14.4 | 5 | 1 | 24 |
| Dubs004_SFMC_26_10_10 | 27,760,516 | 1.18 | 63.9 | 2 | 2 | 17.8 |
| Dubs005_SFMC_17_11_10 | 106,947 | 2.95 | 29.4 | 31 | 5 | 4.5 |
| Dubs006_SFMC_2_12_10 | 1,723,048 | 0.49 | 64.7 | 3 | 12 | 8.8 |
| Dubs001_SFMC_25_5_10_rep | 8,907,141 | 0.06 | 42.1 | 32 | 13 | 8.1 |
| Dubs002_SFMC_14_9_10_rep | 23,343,719 | 0.09 | 58.1 | 1 | 7 | 10.7 |

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Aligning” means a method of comparing a test sequence, such as a sequence read, to one or more reference sequences to determine which reference sequence or which portion of a reference sequence is closest based on some sequence distance measure. An exemplary method of aligning nucleotide sequences is the Smith Waterman algorithm. Distance measures may include Hamming distance, Levenshtein distance, or the like. Distance measures may include a component related to the quality values of nucleotides of the sequences being compared.

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Clonotype” means a recombined nucleotide sequence of a T cell or B cell encoding a T cell receptor (TCR) or B cell receptor (BCR), or a portion thereof. In one aspect, a collection of all the distinct clonotypes of a population of lymphocytes of an individual is a repertoire of such population, e.g. Arstila et al, Science, 286: 958-961 (1999); Yassai et al, Immunogenetics, 61: 493-502 (2009); Kedzierska et al, Mol. Immunol., 45(3): 607-618 (2008); and the like. As used herein, “clonotype profile,” or “repertoire profile,” is a tabulation of clonotypes of a sample of T cells and/or B cells (such as a peripheral blood sample containing such cells) that includes substantially all of the repertoire's clonotypes and their relative abundances. “Clonotype profile,” “repertoire profile,” and “repertoire” are used herein interchangeably. (That is, the term “repertoire,” as discussed more fully below, means a repertoire measured from a sample of lymphocytes). In one aspect of the invention, clonotypes comprise portions of an immunoglobulin heavy chain (IgH) or a TCRβ chain. In other aspects of the invention, clonotypes may be based on other recombined molecules, such as immunoglobulin light chains or TCRα chains, or portions thereof.

“Coalescing” means treating two candidate clonotypes with sequence differences as the same by determining that such differences are due to experimental or measurement error and not due to genuine biological differences. In one aspect, a sequence of a higher frequency candidate clonotype is compared to that of a lower frequency candidate clonotype and if predetermined criteria are satisfied then the number of lower frequency candidate clonotypes is added to that of the higher frequency candidate clonotype and the lower frequency candidate clonotype is thereafter disregarded. That is, the read counts associated with the lower frequency candidate clonotype are added to those of the higher frequency candidate clonotype.

“Complementarity determining regions” (CDRs) mean regions of an immunoglobulin (i.e., antibody) or T cell receptor where the molecule complements an antigen's conformation, thereby determining the molecules specificity and contact with a specific antigen. T cell receptors and immunoglobulins each have three CDRs: CDR1 and CDR2 are found in the variable (V) domain, and CDR3 includes some of V, all of diverse (D) (heavy chains only) and joint (J), and some of the constant (C) domains.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 mL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Quality score” means a measure of the probability that a base assignment at a particular sequence location is correct. A variety methods are well known to those of ordinary skill for calculating quality scores for particular circumstances, such as, for bases called as a result of different sequencing chemistries, detection systems, base-calling algorithms, and so on. Generally, quality score values are monotonically related to probabilities of correct base calling. For example, a quality score, or Q, of 10 may mean that there is a 90 percent chance that a base is called correctly, a Q of 20 may mean that there is a 99 percent chance that a base is called correctly, and so on. For some sequencing platforms, particularly those using sequencing-by-synthesis chemistries, average quality scores decrease as a function of sequence read length, so that quality scores at the beginning of a sequence read are higher than those at the end of a sequence read, such declines being due to phenomena such as incomplete extensions, carry forward extensions, loss of template, loss of polymerase, capping failures, deprotection failures, and the like.

“Repertoire”, or “immune repertoire”, means a set of distinct recombined nucleotide sequences that encode T cell receptors (TCRs) or B cell receptors (BCRs), or fragments thereof, respectively, from a population of lymphocytes of an individual, wherein the nucleotide sequences of the set have a one-to-one correspondence with distinct lymphocytes or their clonal subpopulations for substantially all of the lymphocytes of a sample. In one aspect, a population of lymphocytes from which a repertoire is determined is taken from one or more tissue samples, such as from one or more blood samples. Such blood samples may comprise whole blood or may come from a faction of a whole blood sample, such as peripheral blood mononuclear cells (PBMCs), prepared using conventional techniques. A member nucleotide sequence of a repertoire is referred to herein as a “clonotype.” In one aspect, clonotypes of a repertoire comprises any segment of nucleic acid common to T cell genomic or transcribed sequences or B cell genomic or transcribed sequences which have undergone somatic recombination during the development of TCRs or BCRs, including normal or aberrant development (e.g. associated with cancers). In particular, such somatic recombination is V(D)J recombination, e.g. Jung et al, Cell, 116: 299-311 (2004); Ramsden et al, Semin. Cancer Biol., 20(4): 254-260 (2010); or the like Such nucleic acid segments may include, but not limited to, any of the following: an immunoglobulin heavy chain (IgH) or subsets thereof (e.g. an IgH variable region, CDR3 region, or the like), incomplete IgH molecules, an immunoglobulin light chain or subsets thereof (e.g. a variable region, CDR region, or the like), T cell receptor a chain or subsets thereof, T cell receptor β chain or subsets thereof (e.g. variable region, CDR3, V(D)J region, or the like), a CDR (including CDR1, CDR2 or CDR3, of either TCRs or BCRs, or combinations of such CDRs), V(D)J regions of either TCRs or BCRs, hypermutated regions of IgH variable regions, or the like. In one aspect, nucleic acid segments defining clonotypes of a repertoire are selected so that their diversity (i.e. the number of distinct nucleic acid sequences in the set) is large enough so that substantially every T cell or B cell (or clone thereof) in an individual is represented by a unique nucleic acid sequence in such repertoire. A particular segment or region of recombined nucleic acids that encode TCRs or BCRs (or a portion of either) may be selected that does not reflect the full diversity of a population of T cells or B cells (i.e., each lymphocyte is not represented by a unique clonotype); however, preferably, segments are selected so that clonotypes do reflect the diversity of the population of T cells and/or B cells from which they are derived. That is, preferably each different clone of a sample has different clonotype. In other aspects of the invention, the population of lymphocytes corresponding to a repertoire may be circulating B cells, or may be circulating T cells, or may be tumor-infiltrating lymphocytes, or may be subpopulations of any of the foregoing populations, including but not limited to, CD4+ T cells, or CD8+ T cells, or other subpopulations defined by other cell surface markers, or the like. Such subpopulations may be acquired by taking samples from particular tissues, e.g. bone marrow, or lymph nodes, or the like, or by sorting or enriching cells from a sample (such as peripheral blood) based on one or more cell surface markers, size, morphology, or the like. In still other aspects, the population of lymphocytes corresponding to a repertoire may be derived from disease tissues, such as a tumor tissue, an infected tissue, or the like. In one embodiment, a repertoire comprising human TCRβ chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶. In another embodiment, a repertoire comprising human IgH chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶. In a particular embodiment, a repertoire of the invention comprises a set of nucleotide sequences encoding substantially all segments of the V(D)J region of an IgH chain. In one aspect, “substantially all” as used herein means every segment having a relative abundance of 0.001 percent or higher; or in another aspect, “substantially all” as used herein means every segment having a relative abundance of 0.0001 percent or higher. In another particular embodiment, a repertoire of the invention comprises a set of nucleotide sequences that encodes substantially all segments of the V(D)J region of a TCRβ chain. In another embodiment, a repertoire of the invention comprises a set of nucleotide sequences having lengths in the range of from 25-200 nucleotides and including segments of the V, D, and J regions of a TCRβ chain. In another embodiment, a repertoire of the invention comprises a set of nucleotide sequences having lengths in the range of from 25-200 nucleotides and including segments of the V, D, and J regions of an IgH chain. In another embodiment, a repertoire of the invention comprises a number of distinct nucleotide sequences that is substantially equivalent to the number of lymphocytes expressing a distinct IgH chain. In another embodiment, a repertoire of the invention comprises a number of distinct nucleotide sequences that is substantially equivalent to the number of lymphocytes expressing a distinct TCRβ chain. In still another embodiment, “substantially equivalent” means that with ninety-nine percent probability a repertoire of nucleotide sequences will include a nucleotide sequence encoding an IgH or TCRβ or portion thereof carried or expressed by every lymphocyte of a population of an individual at a frequency of 0.001 percent or greater. In still another embodiment, “substantially equivalent” means that with ninety-nine percent probability a repertoire of nucleotide sequences will include a nucleotide sequence encoding an IgH or TCRβ or portion thereof carried or expressed by every lymphocyte present at a frequency of 0.0001 percent or greater. The sets of clonotypes described in the foregoing two sentences are sometimes referred to herein as representing the “full repertoire” of IgH and/or TCRβ sequences. As mentioned above, when measuring or generating a clonotype profile (or repertoire profile), a sufficiently large sample of lymphocytes is obtained so that such profile provides a reasonably accurate representation of a repertoire for a particular application. In one aspect, samples comprising from 10⁵ to 10⁷ lymphocytes are employed, especially when obtained from peripheral blood samples of from 1-10 mL.

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

“Sequence tree” means a tree data structure for representing nucleotide sequences. In one aspect, a tree data structure of the invention is a rooted directed tree comprising nodes and edges that do not include cycles, or cyclical pathways. Edges from nodes of tree data structures of the invention are usually ordered. Nodes and/or edges are structures that may contain, or be associated with, a value. Each node in a tree has zero or more child nodes, which by convention are shown below it in the tree. A node that has a child is called the child's parent node. A node has at most one parent. Nodes that do not have any children are called leaf nodes. The topmost node in a tree is called the root node. Being the topmost node, the root node will not have parents. It is the node at which operations on the tree commonly begin (although some algorithms begin with the leaf nodes and work up ending at the root). All other nodes can be reached from it by following edges or links.

What is claimed is:

1. A method of screening patients suffering from an autoimmune disease for responsiveness to treatment with a disease modifying anti-rheumatic drug (DMARD), the method comprising the steps of:
determining an IgH clonotype profile from B-cells in a sample of tissue affected by the autoimmune disease, the IgH clonotype profile including IgH clonotypes, IgG clonotypes, and IgD clonotypes; and
classifying a patient as being more likely to respond to DMARD treatment, whenever the patient has, with respect to reference levels characteristic of normal tissue, elevated IgH concentration, elevated IgG fraction, and reduced IgD fraction.

2. The method of claim 1 wherein said elevated level of IgH concentration is at least twice said reference level.

3. The method of claim 1 wherein said elevated IgG fraction is at least twice said reference level.

4. The method of claim 1 wherein said reduced IgD fraction is at least 10-fold less than said reference level.

5. The method of claim 1 wherein said IgH clonotype profile further includes IgM clonotypes and wherein said step of classifying further includes classifying said patient as being more likely to respond to DMARD treatment, whenever said patient further has, with respect to said reference levels characteristic of normal tissue, an elevated IgM somatic mutation rate.

6. The method of claim 5 wherein said elevated IgM somatic mutation rate is at least twice said reference level.

7. The method of claim 1 wherein said step of classifying further includes classifying said patient as being more likely to respond to DMARD treatment, whenever said patient further has, with respect to said reference levels characteristic of normal tissue, a reduced IgD diversity and a reduced IgM diversity.

8. The method of claim 7 wherein a measure of said IgD diversity is a number of different IgD clonotypes in a highest ten percent of frequencies of IgD clonotypes.

9. The method of claim 8 wherein said reduced IgD diversity is less than twenty-five percent of said reference level.

10. The method of claim 7 wherein a measure of said IgM diversity is a number of different IgM clonotypes in a highest twenty-five percent of frequencies of IgM clonotypes.

11. The method of claim 10 wherein said reduced IgM diversity is less than ten percent of said reference level.

12. The method of claims 1 through 11 wherein said normal tissue is peripheral blood mononuclear cells.

13. The method of claim 12 wherein said autoimmune disease is psoriatic arthritis and wherein said tissue affected by said autoimmune disease is synovial fluid.

14. The method of 1 wherein said autoimmune disease is systemic lupus erythematosis.

15. A method of determining responsiveness of a patient having psoriatic arthritis to treatment with a disease modifying anti-rheumatic drug (DMARD), the method comprising the steps of:
determining an IgH clonotype profile from B-cells in a sample of synovial fluid, the IgH clonotype profile including IgH clonotypes, IgG clonotypes, and IgD clonotypes; and
classifying a patient as being more likely to respond to DMARD treatment, whenever the patient has, with respect to reference levels characteristic of normal tissue, elevated IgH concentration, elevated IgG fraction, and reduced IgD fraction.

16. The method of claim 15 wherein said elevated level of IgH concentration is at least twice said reference level.

17. The method of claim 15 wherein said elevated IgG fraction is at least twice said reference level.

18. The method of claim 15 wherein said reduced IgD fraction is at least 10-fold less than said reference level.

19. The method of claim 15 wherein said IgH clonotype profile further includes IgM clonotypes and wherein said step of classifying further includes classifying said patient as being more likely to respond to DMARD treatment, whenever said patient further has, with respect to said reference levels characteristic of normal tissue, an elevated IgM somatic mutation rate.

20. The method of claim 19 wherein said elevated IgM somatic mutation rate is at least twice said reference level.

21. The method of claim 15 wherein said step of classifying further includes classifying said patient as being more likely to respond to DMARD treatment, whenever said patient further has, with respect to said reference levels characteristic of normal tissue, a reduced IgD diversity and a reduced IgM diversity.

22. The method of claim 21 wherein a measure of said IgD diversity is a number of different IgD clonotypes in a highest ten percent of frequencies of IgD clonotypes.

23. The method of claim 22 wherein said reduced IgD diversity is less than twenty-five percent of said reference level.

24. The method of claim 21 wherein a measure of said IgM diversity is a number of different IgM clonotypes in a highest twenty-five percent of frequencies of IgM clonotypes.

25. The method of claim 24 wherein said reduced IgM diversity is less than ten percent of said reference level.

26. The method of claims 15 through 25 wherein said normal tissue is peripheral blood mononuclear cells.

27. The method of claim 15 wherein said IgH clonotype profile indicates at least two of the following conditions hold for said patient: with respect to reference levels characteristic of peripheral blood mononuclear cells, (a) elevated IgH concentration, (b) elevated IgG fraction, (c) reduced IgD fraction, (d) reduced IgD diversity, (e) reduced IgM diversity, and (f) elevated IgM somatic mutation rate.

28. The method of claim 15 wherein said IgH clonotype profile indicates at least three of the following conditions hold for said patient: with respect to reference levels characteristic of peripheral blood mononuclear cells, (a) elevated IgH concentration, (b) elevated IgG fraction, (c) reduced IgD fraction, (d) reduced IgD diversity, (e) reduced IgM diversity, and (f) elevated IgM somatic mutation rate.
